(19) United States
(12) Patent Application Publication
(10) Pub. No.: US 2025/0095815 A1
(43) Pub. Date: Mar. 20, 2025

(54) HEALTH INSURANCE MEDICAL LETTER WIZARD

(71) Applicant: Access Pointe LLC, Amherst, NH (US)

(72) Inventor: Eric Mickelson Mensh, Haverhill, MA (US)

(21) Appl. No.: 18/891,581

(22) Filed: Sep. 20, 2024

Related U.S. Application Data
(60) Provisional application No. 63/584,031, filed on Sep. 20, 2023.

(51) Int. Cl. G16H 20/10; G06Q 40/08; H04L 9/40

(52) U.S. Cl. CPC G16H 20/10; G06Q 40/08; H04L 63/105

(57) ABSTRACT

The present invention relates generally to health care prescription authorization. Particularly, the present invention relates to a method of preparing a prior authorization request, an appeal, or request for formulary exception for prescriptions for a patient capable of being tailored for a specific pharmaceutical with a specific utilization management criteria, with a verification protocol with an optimization protocol, for a specific pharmaceutical, for a specific purpose, with specific tips to address concerns of the insurers' compliance programs.

### RELATED APPLICATIONS

This application claims benefit to a provisional application No. 63/584,031, filed on Sep. 20, 2023.

### COPYRIGHTS RETAINED

The following patent disclosure may contain material subject to copyright protection 2023, and all copyrights thereto are reserved. Notwithstanding the same, the copyright owner has no objection to, and hereby grants a limited license for, the facsimile reproduction by anyone, of the patent document or the patent disclosure, as it appears in the United States Patent and Trademark Office patent files or records.

### BACKGROUND OF THE INVENTION

### 1. Field of the Invention

The present invention relates generally to data processing for health care prescription authorization. Particularly, the present invention relates to a method of preparing a letter of medical necessity, an appeal, or request for formulary exception for prescriptions for a patient.

### 2. Description of the Prior Art

United States Patent Application Publication No. US20120185270A1, by Matthew A. Scantland discloses an apparatus and a method for processing prior authorizations for prescriptions drugs.

United States Patent Application Publication No. US20150058030A1, by Matthew A. Scantland discloses a method of processing a request pertaining to a prescription drug.

Several companies have focused on improving provider efficiency which include: CoverMyMeds, GoodRx for Healthcare Professionals, and WellSky Care Trend. These organizations support provider efficiency by leveraging a single digital platform to simplify the prior authorization requests for a large number of insurance companies.

### SUMMARY OF THE INVENTION

### Advantages and Differences of Invention Over Known Prior Art

While the prior art makes attempts to achieve provider efficiency for the administrative process, none of them enable customizing the program to enable efficiency for a specific pharmaceutical, placing the financial cost for upgrade on the pharmaceutical manufacturer while yet maintaining patient confidentiality and HIPAA compliance to the strictest degree.

There are numerous examples of however, letter templates on numerous drug and device manufacturer websites, however none of them facilitate a method of tailoring a specific type of letter for a specific patient, with a specific history, with specific symptoms, for a specific pharmaceutical, for a specific purpose, with specific tips to address specific concerns of the specific insurance's specific coverage programs.

There are numerous examples of letter templates enabling editing, however none of them facilitate a method of reducing the options thereby tailoring a letter for a specific drug, with a specific utilization management criteria, with a verification protocol with an optimization protocol, for a specific pharmaceutical, for a specific purpose, with specific tips to address concerns of the insurers' compliance programs.

There are several key issues and problems that have been noted with prior art methods. 1) Increasing payer restrictions often require providers to justify treatments. 2) Letter writing is a hassle and time-intensive for providers. 3) Letter writing is often too complicated and difficult to delegate to staff. 4) Current letter templates that are available may be helpful, but due to overall training requirements and implementation protocols, these prior templates are not actually effective at reducing time or hassle involved with letter creation. 5) Letter utilization is difficult to capture. 6) Letter library management is daunting to most physicians and providers, as well as drug and device manufacturers.

FIG. 1A generally illustrates one of the difficulties that has made it impossible for previous methods to address all of these various complaints, problems, and issues with the prior art methods. Specifically, this shared difficulty is one that patients, providers, physicians, manufacturers, and insurance companies all face today, and is one that society in general faces, that of an epidemic of infobesity, i.e., information overload. Patients have information, physicians have information, insurance companies have information, professional organizations have information, compliance programs have information, laboratories have information, government agencies have information, bureaucracies have information, and pharmaceutical companies have yet more information. Each set of information from each of these individual sources represents hundreds, and sometimes, hundreds of thousands of pieces of data. Nor are the numbers of each of these individual sources small and manageable.

According to the US Census Bureau and other industry statistics within the United States, as of 2023, there are over 345 million patients, over 1.1 million active physicians, over 400,000 insurance brokers & agencies businesses, over 20,000 different prescription drug products, over 10,000 professional organizations, and over 88,000 compliance regulations, with approximately 14 billion laboratory tests performed each year. These initial statistics alone are overwhelming, not to mention the actual management and compilation of intersecting and contradicting components required in the final document.

There is a glut of information, but there was previously no easy way to make that information easily available, easily accessible, easily comprehensible, easily organized, and relevant to a particular requirement that needs to be met. This is because the creation of a letter of medical necessity, an appeal, or request for formulary exception for prescriptions for a patient is based upon a unique combination of factors from each of these potential sources. Where the number of unique combinations of m, n, o, p requirements with r categories is determined by the formula: C ((m,n,o,p),r)=n!(n−r)!+o!(o−r)!+m!(m−r)!+p!(p−r)! resulting in an almost infinite number of possible combinations.

For this reason, in many ways, FIG. 1 does not even begin to represent the true chaotic nature of the various sources of data and the errors that accumulate from current methods of trying to compile this data. Further complicating matters, the systems of each of these patients, physicians, insurance companies, drugs, medical products, laboratory tests, professional organizations, are stored on different various platforms that are not designed to interface with each other which makes bringing this information together in a cohesive manner challenging to overcome.

The present method addresses all the issues with the prior art by identifying the problem and turning at least part of the problem into part of the solution, specifically by providing a reiterative method of reducing the possible combinations in a time-efficient manner. By doing this, the present invention is able to provide an apparatus, a method, a system, a computer implemented system, and a finished customizable letter product capable of meeting these unanswered needs of the market. The present invention provides a system to streamline and improve access letter writing to save providers' time and help patients get needed therapies.

The present invention provides an apparatus, a method, and a system to receive a request to generate a request form for a specific prescription drug, select the appropriate type of form from a plurality of form types based on the request type, gather the appropriate type of required details, compile the required details from various databases, verify the patient data provided, optimize the patient data provided, and complete the selected authorization request form for a user so that the user can complete and submit the authorization request form to the health plan and receive a determination on the request.

The present invention was developed to solve the problem of prescribing physicians who were being overburdened by the process insurance companies put in place to assess whether or not access to a prescribed product would be granted. One objective of the present invention is to lower the prescriber's level of burden or hassle related to writing these letters. Another objective of the present invention is to support the efforts of pharmaceutical and medical device manufacturers to improve patient access to medical care by reducing the burden on the prescriber of achieving patient access to their product.

Prior art methods require writing each letter from scratch or possibly in some cases to use prior letters as examples of what to include in new letters of appeal, exception, or medical necessity. The present invention improves over these prior art methods by providing a consistent means to access (and manage) letter templates and provides a systematic process to complete the information that represents the patient specific medical rationale for requesting a specific therapy.

A major problem of the prior art methodology is that the required paperwork also represents a burden which is time-consuming and produces inconsistent results as this burden routinely prevents patients from accessing medically important therapies. The present invention improves over these prior art methods by providing a consistent means to improve the speed, efficiency, and quality of the letters being generated facilitating access for patients to medically important therapies.

The present invention provides a system that leverages a digital wizard-style (sequential step-by-step input) platform that providers can use to generate letters of appeal, exception, and medical necessity in a way that improves efficiency, reduces time spent, and improves quality and consistency. The present invention leverages a digital platform that offers suggested, customizable language and in a well-designed structure so that the user can select specific language and entries rather than create them from scratch for each situation that arises. In some cases, the increased time efficiency per document is improved as much as ten times just due to reduced time associated with typing. If the amount of research previously needed is included in the estimated time for letter creation, the system increases overall time efficiency by over a hundred times compared to previous methods.

The present invention provides a method of creating a request pertaining to a prescription drug. The method includes drafting an appeal, after a claim requesting at least partial insurance coverage of a cost associated with the prescription drug has been submitted to an insurance benefits manager, a notification that prior authorization has been denied. A secured computer system is provided for enabling the method.

The present invention provides an access letter writing platform that simplifies and improves the letter writing process and content allowing providers and their staff to quickly develop effective patient-specific access letters. The present invention has a proprietary, secure web-based platform hosted in a HIPAA and HITECH compliant environment with fully managed firewalls. Strict user controls merge seamlessly with continuous third-party monitoring for security. Business rules and customer service resources are designed to support and manage office-level needs and technical support. Intuitive series of customized prompts result in a comprehensive and customized access letter. Practice information (prescribers, practice logo, etc.) is saved during the account creation process to further simplify letter writing process Letters can be e-signed by the provider to further expedite the creation process.

The present system accommodates unlimited number and length of letter templates. Clinical information is tailored to disease state and brand specific inputs such as ICD10 code(s), laboratory results, etc. For EPIC users, information can be captured from EHR. The present system also provides the ability to copy and paste information from other EHR platforms or patient related materials. Attachments may be included with access letters, such as prescribing information or clinical trials. Data collection may be anonymized to understand letter utilization and support ongoing optimization of access letter library.

The present invention provides a method not only of obtaining the required information from multiple sources, but also of ensuring that only particularly relevant data is available for inclusion into the final letter product by optimizing and restraining the data entry options available to each user.

The present invention achieves this and other objectives by providing a computer-implemented method having the steps of receiving, by a secured server, a first communication comprising first attempt to access secured data by a first user via a first interface over a secured communications network. Analyzing, by the secured server, first user authorization by comparing first user credentials to authorized user credentials stored on the secured server. Optimizing, by the secured server, a plurality of data entry options available to the first user for entering data through a reiterative restraining process.

Specifically, this optimization process includes the steps of identifying targets in each of the plurality of categories as key data targets. Prompting a first data entry by the first user into a first data entry option of the plurality of data entry options, the first data entry option being of a first category of a plurality of categories. Evaluating entered first data entry by comparing the first data entry to the identified targets. Constraining a remainder of the plurality of data entry options associated with a remainder of the plurality of categories according to the first entered identified target. Prompting a next data entry by the first user into a next data entry option of the plurality of data entry options, the next data entry option being of a next category of a plurality of categories. Evaluating the entered next data entry by comparing the next data entry to the identified targets. Then constraining a remainder of the plurality of data entry options associated with a remainder of the plurality of categories according to the next entered identified target.

The step of analyzing, by the secured server, the first user credentials may further involve determining by the secured server, a classification level of the first user classification level by comparing first user credentials to a plurality of authorized user classification levels stored on the secured server.

The optimization process may further involve associating each data entry option of the plurality of data entry options with a classification level of the plurality of authorized user classification levels stored on the secured server.

The optimization process may further involve populating the first and next data entry options of the plurality of data entry options by comparing first user classification level credentials to the plurality of authorized classification levels associated with each data entry option of the plurality of data entry options.

The optimization process may further involve associating each of the plurality of data entry options with at least a first, a second, and a third classification level; determining a classification level of the first user by comparing first user credentials to a plurality of authorized user classification levels stored on the secured server; populating the first and next data entry options of the plurality of data entry options by comparing first user classification level credentials to the plurality of authorized classification levels associated with each data entry option of the plurality of data entry options, and constraining the plurality of data entry options to those associated the classification level of the first user; and prompting a next user with another classification level to begin data entry.

The optimization process may further involve automatically populating a first set of the constrained remainder of the plurality of data entry options based upon a second set of the constrained remainder of the plurality of data entry options; wherein the first set being a first constrained category of the constrained remainder of the plurality of categories; and wherein the second set being a second constrained category of the constrained remainder of the plurality of categories.

The optimization process may further involve synthesizing rationale statements from an amalgamation of a first set of the constrained remainder of the plurality of data entry options, a second set of the constrained remainder of the plurality of data entry options, and a third set of the constrained remainder of the plurality of data entry options.

Each of the categories are contain different types of information: pharmaceutical information, patient information, patient diagnosis information, patient history information, provider information, letter purpose information, proprietary statement information, insurance company information, manufacturer information, professional society information, federal drug administration label dosage information, insurance coverage information, security information, compliance information, state regulation information, federal regulation information, and federal guideline information.

Each of the classifications is a classification of tiered access levels depending upon the user's identity: nurses, administrative assistants, physician assistants, physicians, prescribing physicians, laboratory assistants, pharmaceutical manufacturer employees, secretaries, and security agents.

### BRIEF DESCRIPTION OF THE DRAWINGS

FIG. 1A is a diagrammatic view of the confusing nature of data transfer present in the prior art.

FIG. 1 is a diagrammatic view of the intro page of the program according to one embodiment of the present invention.

FIG. 2 is a diagrammatic view of the registration page of the program of the embodiment shown in FIG. 1.

FIG. 3 is a diagrammatic view of the starting page of the program of the embodiment shown in FIG. 1.

FIG. 4 is a diagrammatic view of the archive page of the program of the embodiment shown in FIG. 1.

FIG. 5 is a diagrammatic view of the settings page of the program of the embodiment shown in FIG. 1.

FIG. 6 is a diagrammatic view of the wizard tips page of the program of the embodiment shown in FIG. 1.

FIG. 7 is a diagrammatic view of the pharmaceutical information page of the program of the embodiment shown in FIG. 1.

FIG. 8 is a diagrammatic view of the new draft page of the program of the embodiment shown in FIG. 1.

FIG. 9 is a diagrammatic view of the provider information page of the program of the embodiment shown in FIG. 1.

FIG. 10 is a diagrammatic view of the patient information page of the program of the embodiment shown in FIG. 1.

FIG. 11 is a diagrammatic view of the draft page of the program of the embodiment shown in FIG. 1.

FIG. 12 is a diagrammatic view of the insurance company information page of the program of the embodiment shown in FIG. 1.

FIG. 13 is a diagrammatic view of the patient diagnosis information page of the program of the embodiment shown in FIG. 1.

FIG. 14 is a diagrammatic view of the patient history information page of the program of the embodiment shown in FIG. 1.

FIG. 15 is a diagrammatic view of the rationale for treatment information page of the program of the embodiment shown in FIG. 1.

FIG. 16 is a diagrammatic view of the final draft page of the program of the embodiment shown in FIG. 1.

FIG. 17 is a diagrammatic view of the final product page of the program of the embodiment shown in FIG. 1.

FIG. 18 is a diagrammatic view of portions of the computer implemented method according to the embodiment shown in FIG. 1.

FIG. 19 is a diagrammatic view of the progress timeline of the embodiment shown in FIG. 1.

FIG. 20 is a diagrammatic view of the system a further embodiment according to the present inventive method.

FIG. 21 is a diagrammatic view of the prompt for data in a first starting category and classification by the system according to the embodiment shown in FIG. 20.

FIG. 22 is a diagrammatic view of the prompt for data in a second creating category by the system according to the embodiment shown in FIG. 20.

FIG. 23 is a diagrammatic view of the prompts for data in a third pharmaceutical category by the system according to the embodiment shown in FIG. 20.

FIG. 24 is a diagrammatic view of the prompts for data in a fourth physician category by the system according to the embodiment shown in FIG. 20.

FIG. 25 is a diagrammatic view of the prompts for data in a fifth patient category by the system according to the embodiment shown in FIG. 20.

FIG. 26 is a diagrammatic view of alternative prompts for data in a fifth patient category by the system according to the embodiment shown in FIG. 20.

FIG. 27 is a diagrammatic view of the prompts for data in a sixth insurance category by the system according to the embodiment shown in FIG. 20.

FIG. 28 is a diagrammatic view of the prompts for data in a seventh diagnosis category with first lab results subcategory, a second ICD-10 subcategory, a third diagnosis subcategory, and an fourth referral information subcategory, by the system according to the embodiment shown in FIG. 20.

FIG. 29 is a diagrammatic view of the prompts for data in an eighth patient history category by the system according to the embodiment shown in FIG. 20.

FIG. 30 is a diagrammatic view of the prompts for data in a ninth rationale for treatment category by the system in the embodiment shown in FIG. 20.

FIG. 31 is the final compilation of data by categories into a final letter product by the system in the embodiment shown in FIG. 20.

FIG. 32 is a diagrammatic view of the alternative prompts for all categories in a letter format by the system in the embodiment shown in FIG. 20.

FIG. 33 is a diagrammatic flow chart of the reiterative restraint process according to the present invention.

FIG. 34 is a diagrammatic flow chart of a further portion of the reiterative restraint process according to the present invention.

### DETAILED DESCRIPTION OF THE INVENTION

The present invention is designed to automate the creation of medical access letters, to reduce the time it takes to write a letter of appeal, exception, or medical necessity, and to improve the quality and consistency of these letters. The present invention does this in one method by providing a program operated on a hosted, HIPAA-compliant server. Account management is handled by a dedicated third-party agency, via a customized service portal. The letter wizard can only be accessed by credentials given to the end user through the medical device or pharmaceutical company.

The letter wizard may be compatible for integration with common electronic health record platforms such as EPIC, Oracle Cerner, and Meditech. For some electronic health record platforms, the letter wizard may provide the user with the ability to copy and paste content. Access may require 2-party authentication to ensure privacy and confidentiality of all medical records.

For ease of referencing across multiple figures and for multiple types of input, is to be understood that the same reference numbers are used to represent both the specific text boxes which are codified for that specific data, and the actual data that is input into those same specific text boxes. As described herein, each specific text box is coded so as to facilitate the automatic import of that specific data from external sources. For instances in which a specific text box has multiple data points which fulfill the codification requirements for that specific data, the multiple data points are visible in drop-down menu format. This specific data for each specific text box may also be entered manually where allowed. In other embodiments, only automatic entry is allowed while manual entry is prohibited in certain text boxes, in order to ensure preservation of secure information which should be retained.

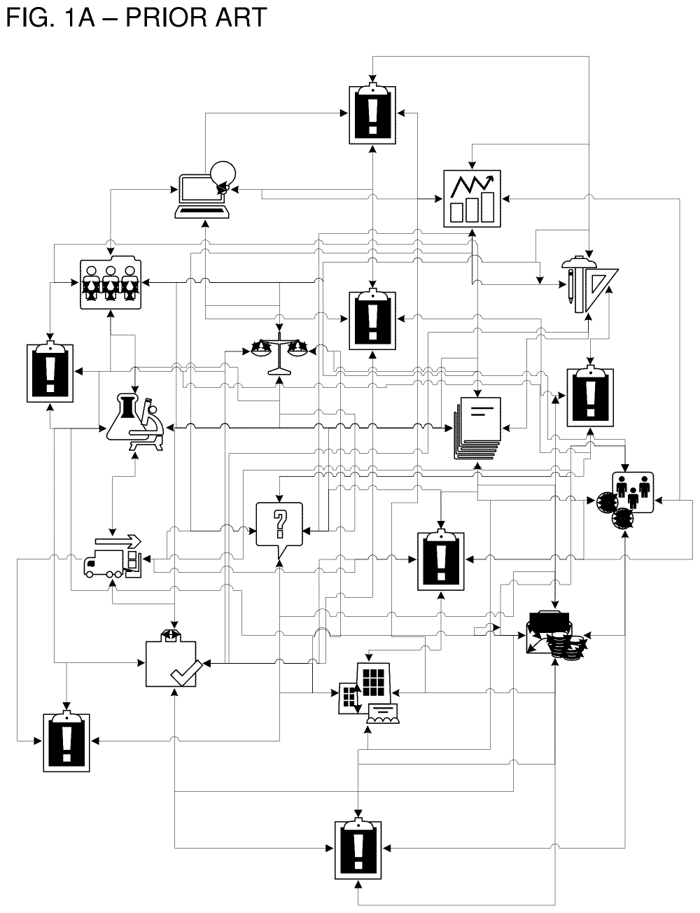

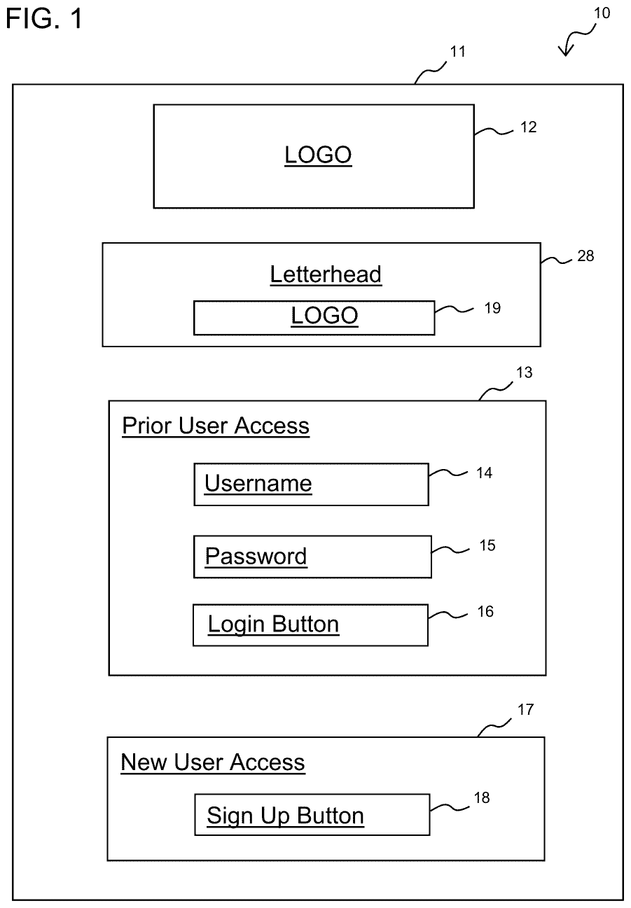

### FIGS. 1-19—Letter Wizard

Portions of a program according to a preferred embodiment of the present invention are diagrammatically illustrated in FIGS. 1-17. Specifically, for discussion of the present inventive method and system, FIGS. 1-17 will refer to diagrammatic representations of screenshots of viewpoints of the program according to one embodiment of the present inventive system. The present inventive method reduces user confusion by providing the data entry options in a user-friendly format which is familiar to the user. Meanwhile, the system optimizes the overall process by automatically providing data entry and reducing the available data entry options depending upon the particular user according to the associated classifications and categories of the data entry text boxes.

### FIGS. 1 & 2—User Access & Registration 20

FIG. 1 is a diagrammatic view of the screenshot of an intro page of the user interface to the system according to one embodiment of the present invention. The initial access page 11 of the system is accessible through a user interface 10 such as a secure personal computer or other security enabled electronic accessible system. The initial access page 11 may provide the company logo 12 with a prior user access section 13 requesting that a user provide their username 14 and password 15, with login button 16 to finalize the request. The new user access 17 section inquires as to whether the end user is a new user and provides a sign-up button 18 which connects to the new user registration page 20. Where applicable, an optional second logo 19 may also be provided.

In some embodiments, the first logo 12 may be provided which is the alterable user's company logo, while the second logo 19 may reflect the system's company logo. In other embodiments, the first logo 12 may be the present inventive system's company logo, while the second logo 19 may reflect the user's company logo. Regardless, the present inventive system's logo will remain unalterable within the program where utilized, while the user's company letterhead 28 may have a logo 19 which may be altered as desired.

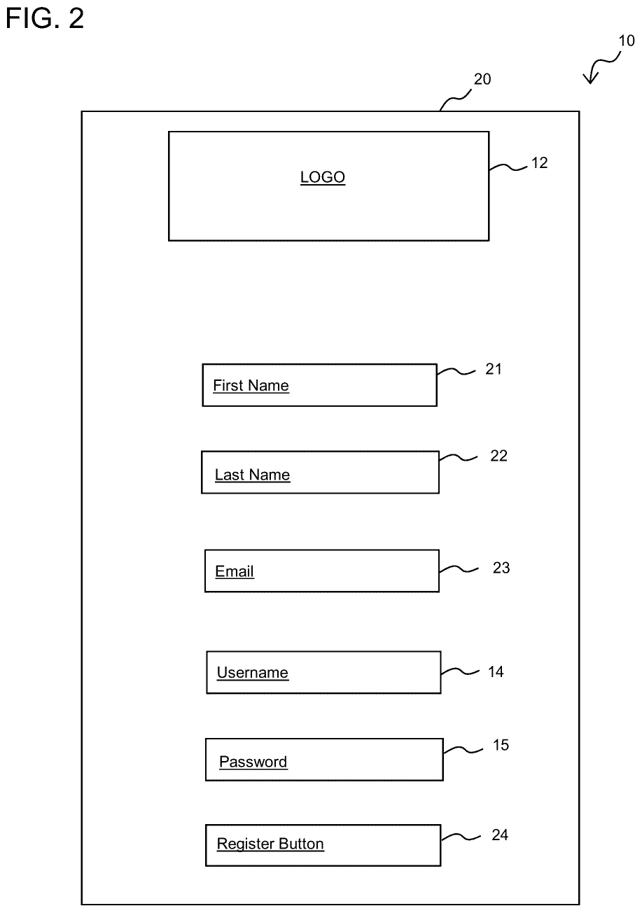

FIG. 2 is a diagrammatic view of the new user registration 20 page of the program of the embodiment shown in FIG. 1. New users provide their first name 21, last name 22, and email 23, while entering their new username 14 and password 15 to register 24. If the information is verified, then the new user is able to log-in to the starting page.

In other embodiments, the practice information may also be input by the first user. This information may include: provider first name, provider last name, provider title (MD, DO, NP, PA), provider NPI #, practice street address #1, practice street address #2, practice city, practice state, practice zip code, and practice logo. If there is more than one provider in the practice, the first name, last name, title, and NPI #for each additional provider may also be entered. This information may be saved to simplify the letter-writing process by automatically populating that section of the letter. If there is more than one provider, the information may be available in the form of a dropdown selection for the provider's first name, last name, title, and NPI #.

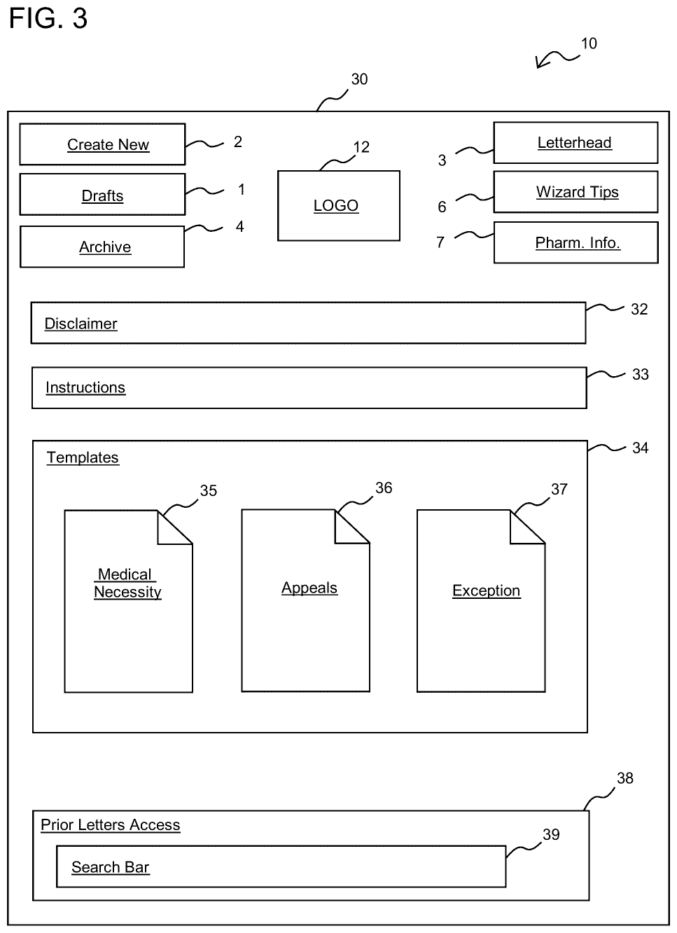

### FIG. 3—Starting 30

FIG. 3 is a diagrammatic view of the starting page of the program of the embodiment shown in FIG. 1. Once the user creates an account, the user will have access to the starting page 30. In one embodiment, links 1, 2, 3, 4, 6, 7 to various other parts or pages are provided at the top of the screen, such as to the button 2 to start a new letter 30, the button 1 to access saved drafts 100, a button 3 to access the company letterhead 28, a button 6 to access the wizard tips 60, and a button to access the pharmaceutical information 70.

The starting page may provide a disclaimer 32, instructions 33, and template types 34. The user can then select from one of the letter types 34, a letter of medical necessity 35, a letter of appeal 36, or a letter requesting a formulary or medical exception 37. The starting page 30 may also include a section for prior letters access 38 with a search bar 39 capable of free form searching through the previous document archive 40 or through the users' external database.

Each user can upload previously created letters for editing. PDFs created through the letter creation platform will include HTML code so they can be edited or reused for subsequent letters. The platform can accommodate an unlimited number of products or types of letters. The number of brands and or letter types will vary based on patient needs.

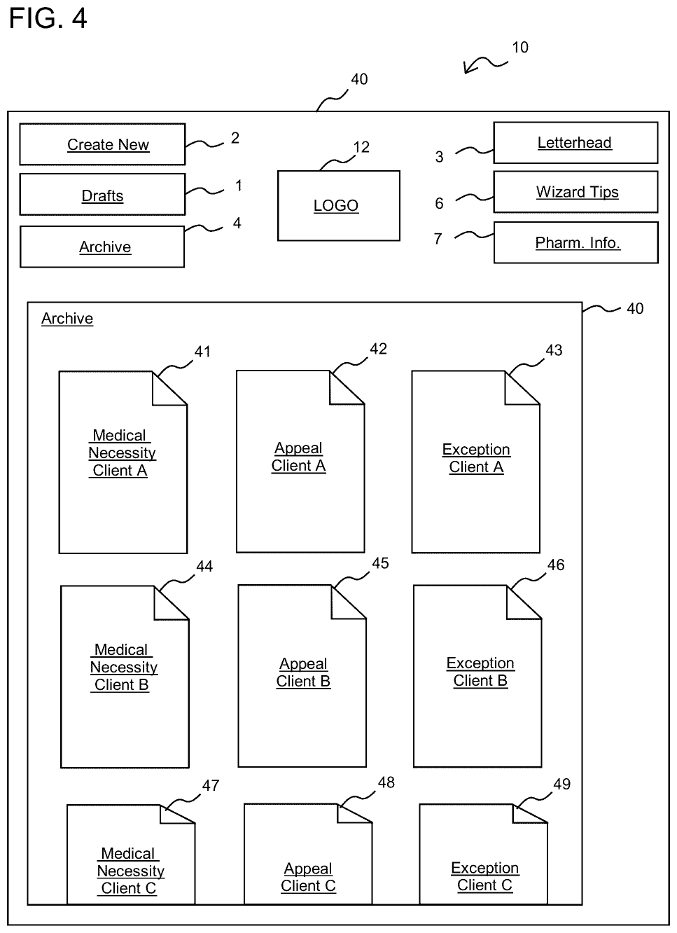

### FIG. 4—Archive 40

FIG. 4 is a diagrammatic view of an archive page 40 of the program of the embodiment shown in FIG. 1. Examples of potential prior letters are shown here in this archive. Shown here are letters written for the first patient A: a first letter 41 which was written for medical necessity; a second letter 42 which was written for appeal; and a third letter 43 which was written for exception. Also shown are letters written for a second patient B: the first letter 44 which was written for medical necessity; a second letter 45 which was written for appeal; and a third letter 46 which was written for exception. Finally, also shown are letters written for a third patient C: the first letter 47 which was written for medical necessity; a second letter 48 which was written for appeal; and a third letter 49 which was written for exception.

In other embodiments, an archive function is not provided in order to reduce the data requirements on the server. In these embodiments, HTML code may be provided which is capable of converting PDFs of previous letters, so these previous letters can be edited or reused for subsequent letters.

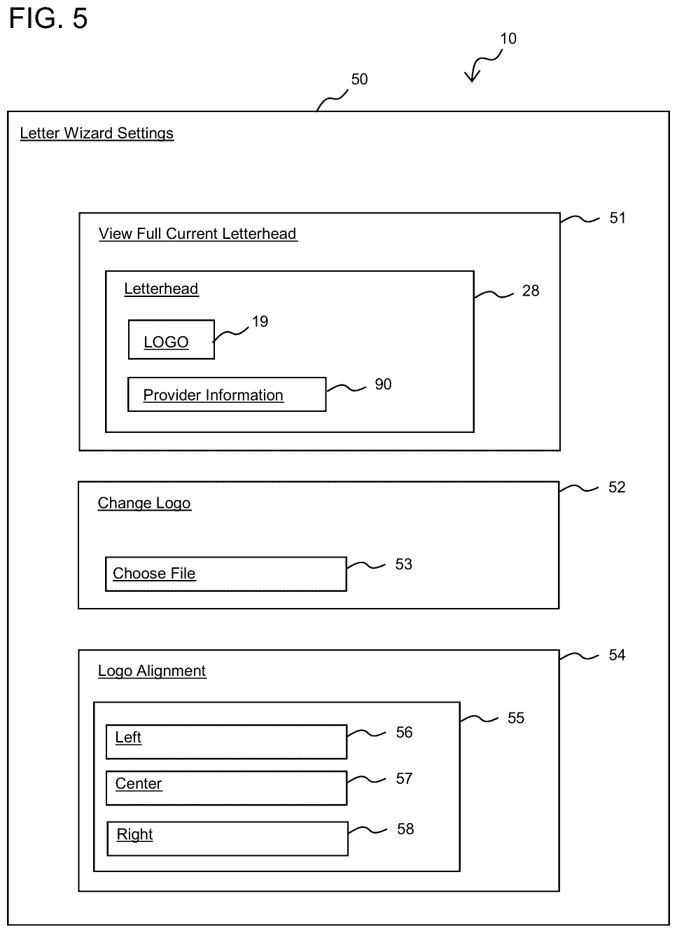

### FIG. 5—Settings 50

Once in the program, the user, often an office admin at this stage, will have the ability to set the system up for all providers' future use, including the addition of a logo, company/practice information such as company name, address, phone, and fax, and will be able to arrange this information in a variety of ways on the page. Once set up, the system will remember the setting and future letters will be started with the same template look and feel.

For example, FIG. 5 is a diagrammatic view of one of the settings pages 50 of the program of the embodiment shown in FIG. 1. The letter wizard settings 50 may enable the user to view current letterhead section 51 which displays the users' current letterhead 28 which may include the current logo 19 and the current provider information 90. A change logo section 52 is provided to facilitate choosing a new logo 53 from a file. Finally, the logo position section 54 may facilitate changing a logo alignment. A dropdown menu 55 may be provided with multiple alignment options: left 56, center 57, and right 58.

In another embodiment, these options will be provided during the initial account registration process for new accounts. For existing users, account information may be edited in the user profile section.

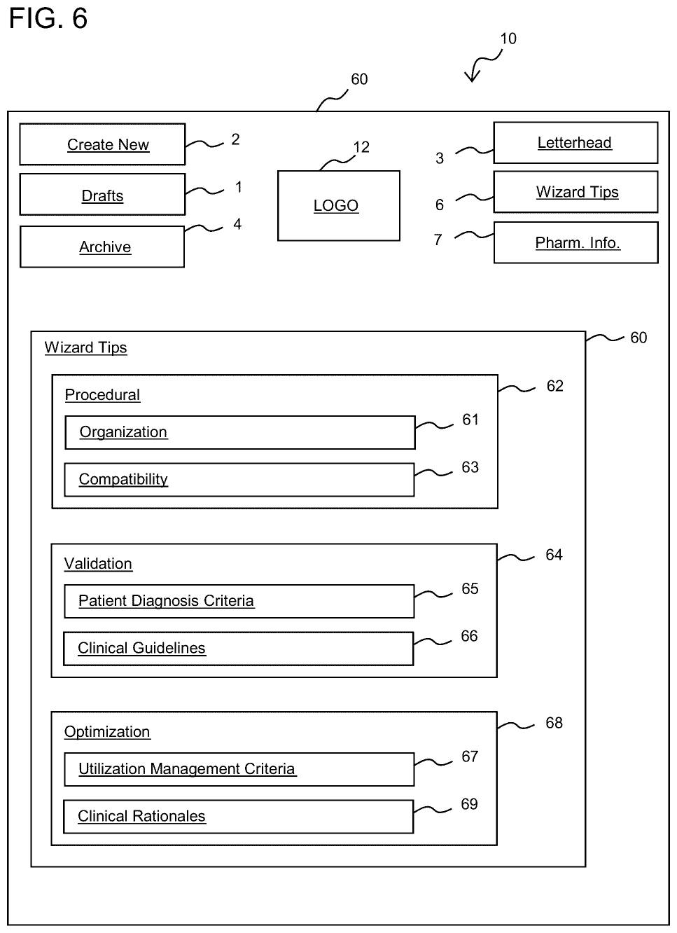

### FIG. 6—Wizard Tips 60

FIG. 6 is a diagrammatic view of the wizard tips page 60 of the program of the embodiment shown in FIG. 1. On each drafting page, there will be a “tips” icon the user can click on to give them insights and examples of the types of information that can be important in this section. Some of these tips are used in the procedural process 62, some in the validation process 64 and others are used in the optimization process 68 described elsewhere herein. In some embodiments, these tips are accessible via a bottom ribbon. In some embodiments, these tips may include a quick start guide having a standard explanation of how to use the program. Further embodiments may include tips which are brand specific, and/or customizable materials for easy access by user.

Procedural tips 62 include, but are not limited to, those that focus on providing tips on organizational information 61 such as the provider information 90 and patient information 110. These tips 62 also include information on system usage such as compatibility issues 63 such as downloading data from electronic records, exporting data, and cross platform compatibility support.

Validation tips 64 include, but are not limited to, data which is downloaded from an electronic record, exported, or provided via cross platform support. The emphasis here is ensuring that all the information provided is accurate information. Ensuring that there is supporting documentation for each statement. Clinical guidelines which provide recommendations on how to diagnose and treat a medical condition. In some embodiments, this may be coded into the letter template prompts or achieved through the inclusion of drop-down menu options. Once an option is selected, the user will be able to edit to ensure the statements are tailored to the patient.

Additional details about the diagnosis are helpful here, as these are generally different from the details that are typically listed in the description of the patient's recent symptoms/conditions. Suggested commentary regarding discussions of family history, and other co-morbidities, are helpful here. For example, if the age of the patient is different from the recommended age, a discussion of the atypical weight would be helpful here. Additional statements from chart notes and other physician notes documenting diagnosis and disease state-specific symptomatology. This section also provides examples of other random specific details from labs (that do not exactly fit one of the other areas).

Referral information including details from primary care physicians, and other specialists are also provided here. Automatic statements “Referral provided by Dr. ______ of ______ on the date of ______” with the blanks being automatically filled in from data download from electronic records make filling in these statements more efficient.

The optimization tips 68 includes, but is not limited to, utilization management criteria 67, and clinical rationales 69. Generally, utilization management criteria 67 are designated, arranged, and organized to ensure proper performance of the program. Utilization management criteria according to the present inventive method includes data on the formulary position, required step therapies, prior treatment requirements, quantity limits, lab and diagnostic test results, trial inclusion criteria, trial exclusion criteria, age limits, and additional diagnostic and disease state-specific symptomatology.

Formulary position data is obtained from databases such as Fingertip formulary and MMIT and generally includes information specific to the medication including: on formulary, tier and co-pay/co-insurance, not on formulary (available through exception), formulary exclusion (not available through exception), and formulary tiers.

Step therapy data is obtained from databases such as Health Plan Coverage and Pharmacy Policy databases, MMIT, and Fingertip Formulary and integrated into the present system when specific to the manufacturer's drug or device and may include information including step therapy protocols involving: generic treatments; alternative/preferred treatment 1; alternative/preferred treatment 2; and alternative/preferred treatment 3.

Related to required step therapy protocols are frequent prior treatment options including non-pharmacologic interventions. These are obtained from sources such as the Health Plan Coverage and Pharmacy Policy databases and professional society guidelines. These options may include: physical therapy, duration/dates; occupational therapy, duration/dates; and medical or surgical interventions (injections, ablations, stents, etc.), type/dates.

Quantity limits are identified as key targets when obtained by the system from outside databases including FDA-labelled dosage, and Health Plan Coverage and Pharmacy Policy. These limits may include differences according to the type of treatment. For chronic therapy, month limits, e.g., thirty oral solids/month or number of injections per month. For reoccurring episodic treatment-limited to labeled course of therapy to prevent off label utilization. For one-time treatments, e.g., gene therapy, these limits are also noted by the system and identified as key targets.

Lab and diagnostic test results are also often required criteria, and the presence of these results fall under the verification process of the present inventive method. Lab and diagnostic test results may include imaging (X-ray, ultrasound, CT scan, MRI, PET scan, fluoroscopy, contrast radiography, etc.); physical measurements (blood pressure, intraocular pressure, contractures, etc.); confirmatory diagnostics (genetic testing, rheumatoid factor, etc.); lab values (blood counts, hormones, titer, antibodies, etc.); and observations (timed testing, activities of daily living, ambulatory status, movement disorders, etc.). Examples include: CRP, Blood counts, LFT, kidney panel, thyroid panel, mental acuity testing, activities of daily living (ADL) functional testing, timed ambulation, pulmonary function testing, EKG, and EEG.

Trial inclusion/exclusion criteria are also often required criteria, and the presence of these results fall under the verification process of the present inventive method. Inclusion examples include: specific blood level, e.g., A1C>6.5%; patient tried and failed pharmacologic or non-pharmacologic therapy, e.g., symptoms present despite use of anti-inflammatory therapy; specific age, e.g., adults (18 or older); and or the presence of specific symptoms may be required to be included in the trial. Exclusion examples include: organ dysfunction, e.g., liver or kidney dysfunction; multiple chronic conditions; older adults, e.g., >age 65; children, infants, and adolescents e.g., <12 months or age or infants; pregnant or lactating women.

As many inclusion and exclusion criteria focus on age, age limits (based on labelled indication) are also identified as key target data in the verification process of the present inventive method. These requirements are obtained by the system and integrated from various outside databases including FDA-labelled dosage, and the relevant Health Plan Coverage and Pharmacy Policy. Age brackets include examples such as: adults: 18 and older; pediatric: under 18 or specific age according to label, e.g., 12 and older; and older adults, e.g., >age 65.

Additional chart notes are often also a part of the optimization process according to the inventive method of the present invention and are incorporated from databases such as the secure electronic medical record. Examples include physician notes documenting diagnosis and disease state-specific symptomatology.

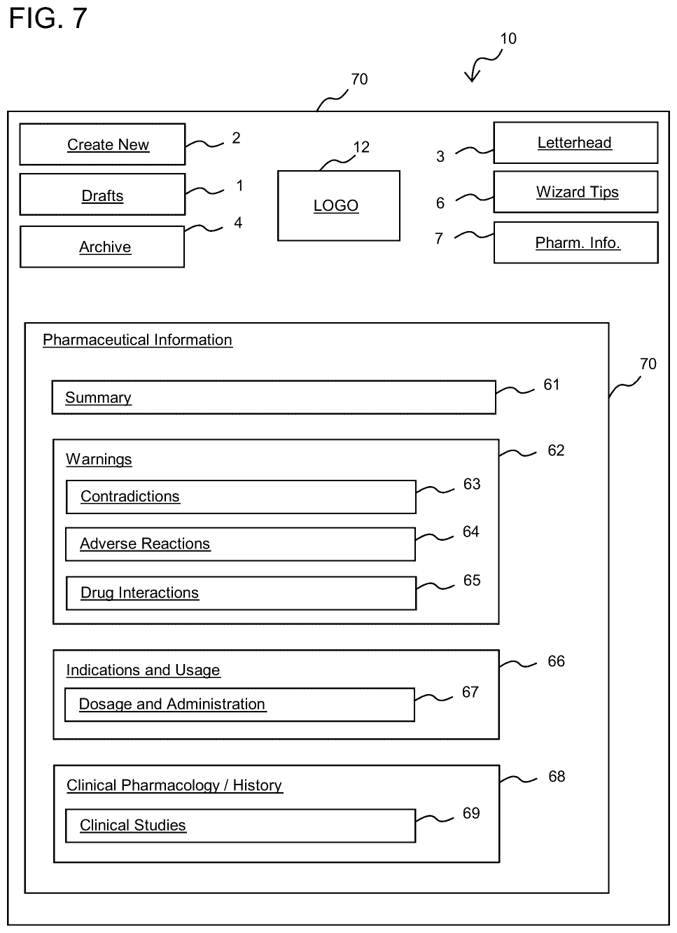

### FIG. 7—Pharmaceutical Information 70

FIG. 7 is a diagrammatic view of the pharmaceutical information page 70 of the program of the embodiment shown in FIG. 1. This pharmaceutical information 70 includes data that, according to the inventive method of the present invention, are incorporated from databases such as the original pharmaceutical manufacturer's package information data sheet. Categories here include a summary 71, warnings 72, indications and usage 76, and clinical pharmacology/history 78 which are specific to the particular pharmaceutical of concern. Warnings 72 may include contradictions 73, adverse reactions 74, and drug interactions 75. Indications and usage 76 may include dosage and administration 77 according use for specific populations. Clinical pharmacology and history 78 further include additional details such as any clinical studies 79 which were relevant.

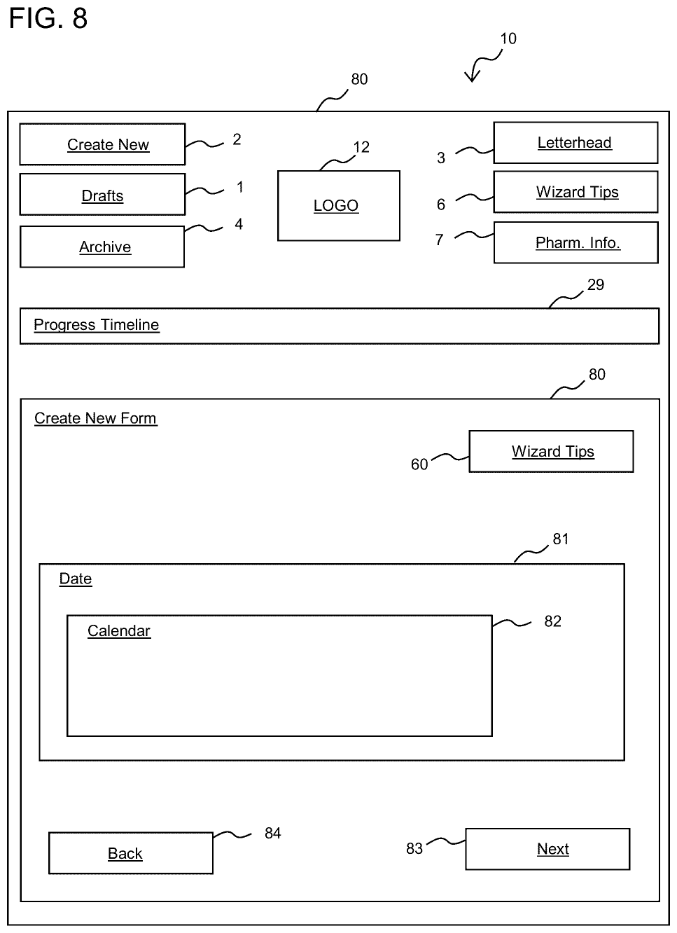

### FIG. 8—Create New Letter 80

FIG. 8 is a diagrammatic view of the new draft page 80 of the program of the embodiment shown in FIG. 1. Links to various pages are provided at the top of most pages of the program, such as the button 2 to start a new letter 30, the button 1 to access saved drafts 100, the button 3 to access the settings for the company letterhead 28, the button 6 to access the wizard tips 60, and a button to access the pharmaceutical information 70. In other embodiments, other links might be made available to pages such as an individual account profile, home, preview, and/or create letter.

A progress timeline 29 showing a progression of each of the sequential steps in the task, process, or workflow is provided along the top, or left side, of the page. Specifically, each of the sequential steps of creating the letter is listed: letter type 34, provider details 90, patient details 110, insurance 120, diagnosis 140, history 190, and rationale 198 (conclusion).

A link to wizard tips 60, specific to this type of template is repeated in the starting page section to encourage users to access them. A first date section 81 provides a dropdown menu 55 with a full calendar 82 enabling users to identify the relevant date for this template. Buttons at the bottom of the page enable a user to go back 84 to a previous page, or forward to the next page 83 along the progress timeline.

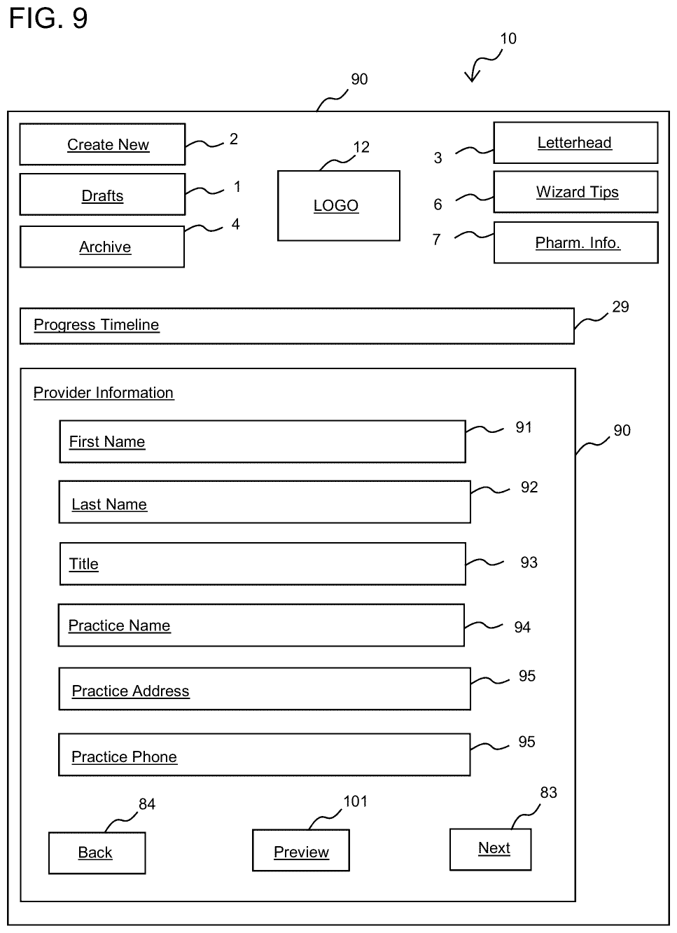

### FIG. 9—Provider Information 90

FIG. 9 is a diagrammatic view of the provider information page 90 of the program of the embodiment shown in FIG. 1. Information for provider first name 91, provider last name 92, provider title 93, provider practice name 94, provider practice address 95, provider practice phone 96, and NPI number, may be provided by the user. In another embodiment, this information is input by the system automatically through saved information in the program, or through automatic input via authorized access of the provider database user profile. In most embodiments, information in this category will be provided during the initial account registration process by office personnel, thus saving the provider time. During this step then, all that is necessary is for the provider to confirm that the information provided is accurate.

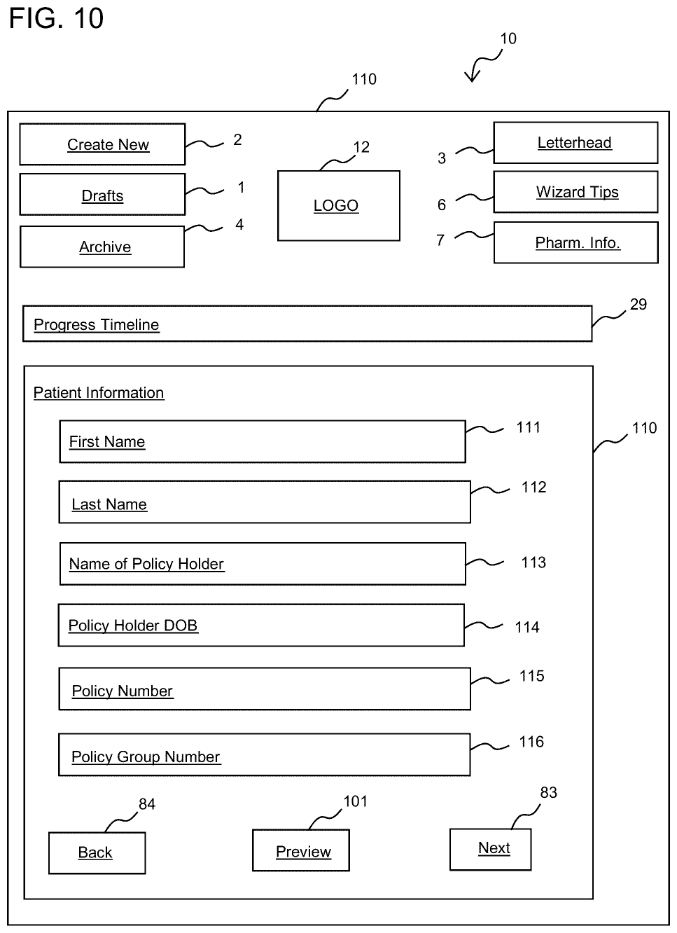

### FIG. 10—Patient Information 110

FIG. 10 is a diagrammatic view of the patient bibliographic information page of the program of the embodiment shown in FIG. 1. Patient information 110 will include data such as patient bibliographic information such as first name 111, last name 112, and date of birth, patient insurance information such as the name of policy holder 113, policy holder DOB 114, policy number 115, and policy group number 116, patient diagnosis 140, and patient history 190. The user may manually provide this information. In another embodiment, this information is input automatically through saved information in the program, or through automatic input via authorized access of the patient database profile. Options are provided to return to the previous page 84, continue to the next page 83, or preview 101 the document.

In some embodiments, this program can automatically obtain patient information. Upon request, the program may query common electronic health record platforms for patient information starting with the input of their name. In a program such as EPIC, closest results are first returned so that the user can review and pick the appropriate patient profile. Thus, ensuring that the proper patient profile is connected to the letter. The user can also cut and paste other information from other common electronic health record platforms. This program is unique in that there is a toggle that can be selected. Specifically, the program queries whether the patient and policy holder are the same people. If they are the same, the information is automatically duplicated in the insurance policy information, which can improve ease and accuracy of letters and avoid discrepancies due to typographical errors.

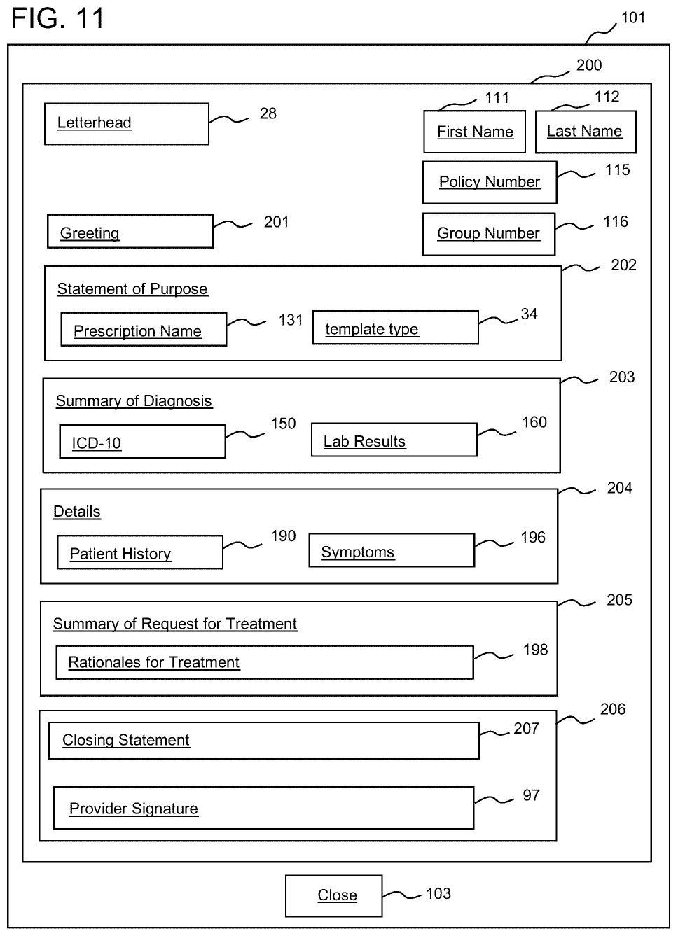

### FIG. 11—Viewing Draft Page 101

FIG. 11 is a diagrammatic view of the draft or preview page 101 of the program of the embodiment shown in FIG. 1. All saved drafts 100 can also be accessed through the buttons on most pages, or a specific draft can be accessed via the review draft button 101. In order to ensure that documents are not sent unfinished, the interim drafts 101 options differ from those of the final draft 102. Specifically, the interim drafts 101 only option is to close 103 the draft and continue drafting the final product, while the final draft 102 has additional options of saving 104 to include yet further details, or to finalize and create the letter 105. In other embodiments, drafts are not saved beyond the preliminary creation date to minimize protected health information capture or storage.

As shown in FIG. 11, the interim draft view page 101 shows a print preview of document 200. The patient information is shown in a designated portion of the document 200 according to specific medicament policy formats and may include data such as first name 111, last name 112, name of policy holder 113, policy holder DOB 114, policy number 115, and policy group number 116. This is retrieved from the patient information as shown in FIG. 10. A standard greeting 201 is provided, followed by a statement of purpose 202 including the prescription name 131 and the template type 34, i.e., a letter of medical necessity 35, a letter of appeal 36, or a letter requesting a formulary or medical exception 37.

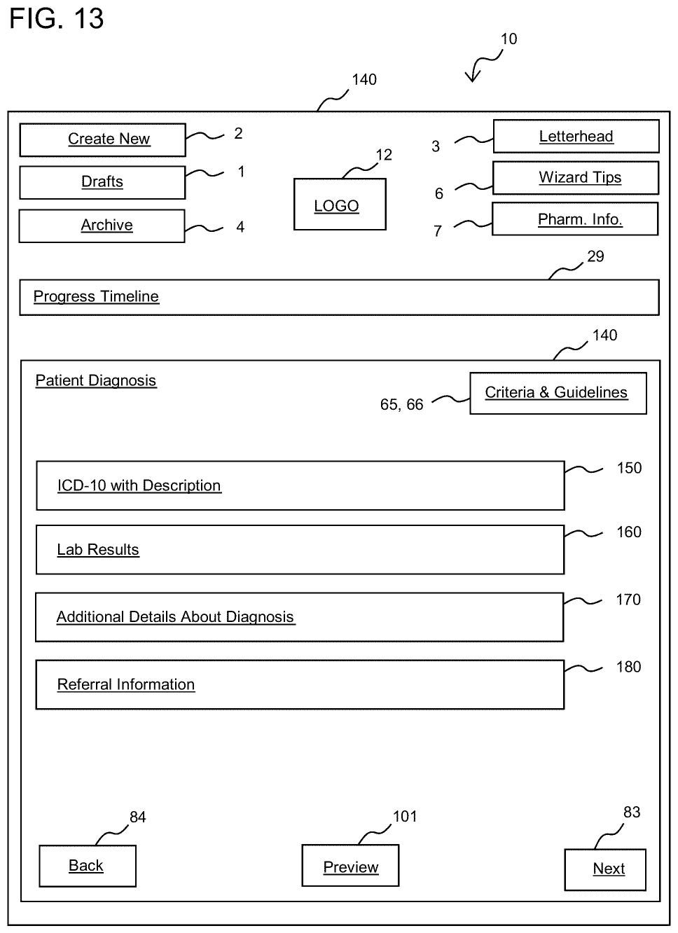

The next section is a summary of the diagnosis 203 including details from the patient diagnosis 203 as discussed in reference to FIG. 13 and will include relevant details from the patient diagnosis 203, such as but not limited to, the ICD-10 150 and lab results 160. The next section thereafter according to some templates will be the further details 204 including the patient history 190 and patient symptoms 196 that support the purpose of the present letter 200. The rationale for treatment section 205 will include tips 60 and rationales 199.

The final section 206 includes a closing statement 207 and provider information such as the provider first name 91, provider last name 92, provider title 93, provider practice name 94, provider practice address 95, and provider practice phone 96. In some embodiments, the closing statement 207 in the final section 206 will include a verified e-signature option connected to the first and last name 91, 92, which facilitates expediency of overall letter creation.

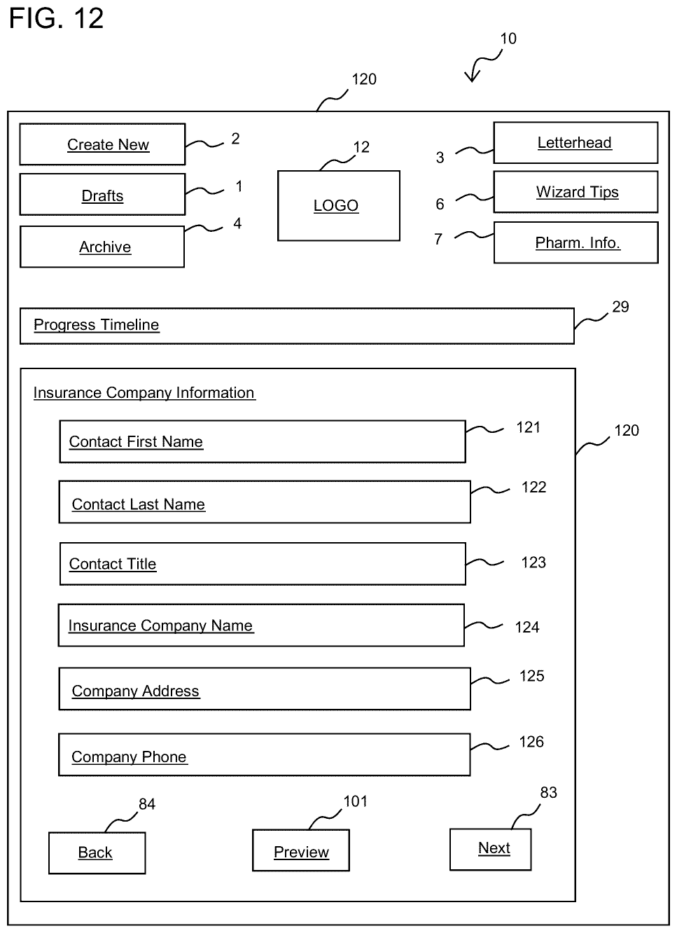

### FIG. 12—Insurance Company 120

FIG. 12 is a diagrammatic view of the insurance information of the program of the embodiment shown in FIG. 1. The insurance information 120 may include contact first name 121, contact last name 122, title 123, company name 124, company address 125, company phone 126, pharmaceutical information 130, and prescription name 131 associated with insurance company 290. In some embodiments, the user may manually provide this information. However, in most embodiments, this information is input automatically through saved information in the program. While in other embodiments, this information may be automatically input via authorized access of the insurance database. Indeed, one of the benefits of the present invention is the capability to provide this information upfront, preloaded, for an optimized, streamlined, and more efficient drafting process from a user perspective.

### FIG. 13—Patient Diagnosis 140

FIG. 13 is a diagrammatic view of the patient diagnosis information page 140 of the program of the embodiment shown in FIG. 1. As before, links to various other pages are provided at the top of most pages of the program, such as the button 2 to start a new letter 30, the button 1 to access saved drafts 100, a button 3 to access the company letterhead 28, a button 6 to access the wizard tips 60, and a button to access the pharmaceutical information 70. Again, a progress timeline 29 showing a progression of each of the sequential steps in the task, process, or workflow is also provided along the top of the page.

Sections for inputting and reviewing imported information are provided for ICD-10 150, lab results 160, additional details about diagnosis 170, and referral information 180 specific to a patient.

The dropdown menu for ICD-10 150 according to the present invention allows details to be visible to the user that are only relevant to the specific prescription 131. Unlike general template wizards that require that the user browse through hundreds of irrelevant options, the present invention filters out irrelevant options to streamline the drafting process.

Lab results 160 may include summary statements such as the results of common lab tests done, e.g., body fluid culture: no growth gram; stain result: no WBCs or organisms seen; C reactive protein: <4.0 mg/L<=10.0 mg/L; magnesium: 2.1 mg/dL 1.6-2.6 mg/dL; and Bilirubin 0.9 mg/dl 0.2-1.2 mg/dL. Additional details about the diagnosis 170 may include, but are not limited to treatment history, test results, and other details related to the severity of the condition. In some embodiments, the program can query common electronic health record platforms for a specific patient to obtain these lab values as well as other key components of patient treatments, history and or chart notes which are all potential requirements of insurance plan requests to prove medical relevance or need. In other embodiments, this information can also be copied and pasted manually from common electronic health record platforms.

A referral section 180 is also provided for inputting the first and last name of the referral contact. In most embodiments, this information is input automatically through saved information in the program, or through automatic input via authorized access of the patient database profile. However, the user may manually input this information. Options are provided to return to the previous page 84, continue to the next page 83, or preview 101 the document.

The patient diagnosis 140 page will include an extra link to specifically relevant wizard tips 60 to provide a verification process. Specifically, the verification process includes providing ‘tips’ as to sample lab results, including lab results and criteria which are necessary or recommended. This gives the physician an opportunity to ensure that all of the necessary tests have been performed before submitting the request.

In some embodiments, if the verification process indicates that a specific lab test is required for authorization of this prescription, then the provider can save the draft 104 and order the necessary tests. Then, when the tests are completed, the physician can return to the drafts 100 section to complete the document 200 with the requisite lab results. This ensures that requests lacking the requirements are not submitted unknowingly, saving each of the patient, insurer, and physician time, effort, and energy by reducing the number of insufficient requests which delay time until the patient can receive the requisite prescription.

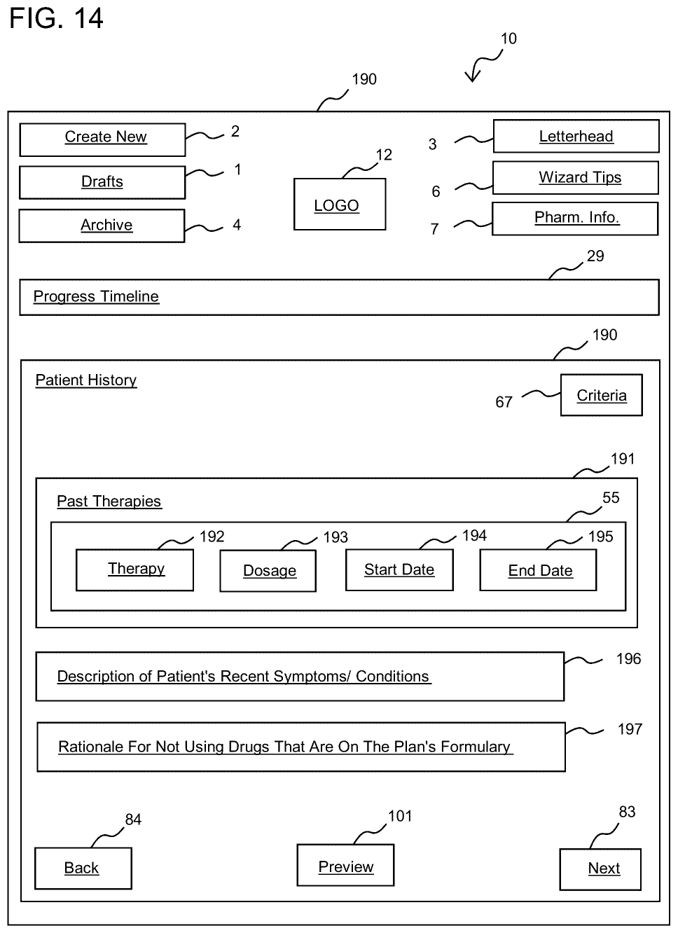

### FIG. 14—Patient History 190

FIG. 14 is a diagrammatic view of the patient history information page of the program of the embodiment shown in FIG. 1. The patient history information page 190 includes sections for past therapies 191, symptoms 196, and rationales for treatment 197. In some embodiments, the system can automatically query common electronic health record platforms for a specific patient to obtain patient history information including past therapies, symptoms, patient treatments, history, chart notes, and rationales which are all potential requirements of insurance plan requests to prove medical relevance or need. In other embodiments, this information can also be copied and pasted manually from common electronic health record platforms.

The past therapies section may include dropdown menus and calendars for therapies 193, dosages, 193, and start dates 194, and end dates 195. For example, for uromysitisis treatment, the past procedures may include a list of potential previous potential therapies such as physical therapy, counseling, erythromycin, levofloxacin, ofloxacin, moxifloxacin, and ciprofloxacin. The dosage dropdown menu 193 could include N/A, 5 mg/kg 1× per week pill form, 200 mg 2× per day pill form, 12 mg 1× per month pill form, 5 mg/kg per quarter year intravenous (IV) infusion, or annual IV infusion. The start date 194 and end date 195 calendars provide easy review and input of the relevant dates.

The patient history 140 page will include an extra link to specifically relevant wizard tips 60 to provide an optimization process to ensure the most optimal outcome. Specifically, the optimization process may include providing examples of the types of information being requested or complete or mostly complete ‘tips’ including sample symptom descriptions, sample common symptom patterns, and sample symptom criteria which are necessary or recommended for this specific prescription.

For example, one such statement for this prescription may be: “Patient presented with [option: frequent, infrequent, rare daily, weekly, monthly,] urinary incontinence which has been controlled for [period of time: _ months] with [option: conservative, aggressive] therapeutic interventions.” The physician could then enter the whole statement “Patient presented with frequent urinary incontinence which has been controlled for periods of time with conservative and more aggressive therapeutic interventions.” within three seconds, as opposed to the 20-40 seconds that it would normally take to type manually.

Again, another such statement for this prescription may be: “The patient has regressed from a maximum improvement of [enter number] incontinence events back to their original presentation of [enter number] events per day.” Using this, within another two to four seconds, the physician could then enter the applicable statement, “The patient has regressed from a maximum improvement of 9 incontinence events back to their original presentation of 15 events per day.” Again, as opposed to the 20-40 seconds that it would normally take to type manually, representing a time savings of approximately 1000 percent, or ten times.

This also provides the additional benefit of giving the physician an opportunity to ensure that all of the relevant symptoms are provided before submitting the request. This ensures that requests lacking the optimum descriptions are not submitted unknowingly, saving each of the patient, insurer, and physician time, effort, and energy by reducing the number of insufficient requests which delay time until the patient can receive the requisite prescription.

Lastly on this screen, the user will be asked to enter rationales for requesting the use of a particular drug or device when it is not on the insurance plan's formulary. This will also be free text but there will be a “sample text” icon on which the user can click to get an example of the types of information being requested. As with previous screens, once completed, the user will be able to preview the partially completed letter.

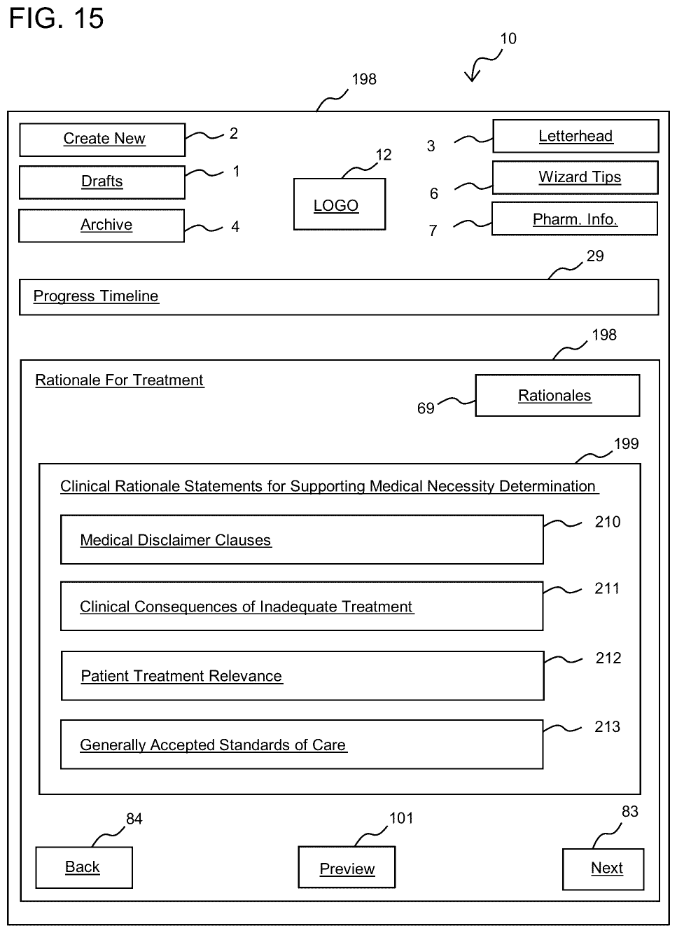

### FIG. 15—Rationales for Treatment 198

FIG. 15 is a diagrammatic view of the summary of the rationales for treatment information page 198 of the program of the embodiment shown in FIG. 1. While a physician is capable of entering these rationales in free form in this section, rationale statements and examples are suggested 69. These rationales 69 are synthesized and provided in dropdown format as part of the reiterative restraint optimization process 68. Generic examples of these statements 198 are provided above with reference to FIG. 14.

The wizard tips 69 provided here are specifically a dropdown menu for inserting clinical rationale statements for supporting medical necessity determination 69. One of the sources for the underlying data in these statements comes from various sources, including databases such as FDA-label dosage, relevant Health Plan Coverage and Pharmacy Policy, and the professional society guidelines. The final statements are an amalgamation of an initial set of statements stored on the memory of the server, then fused with the restrained data feedback received from each of the other categories. Examples of these statements 197 may include the following:
Medical disclaimer clauses 210 are an amalgamation of sorted and restrained initial statements, patient information, past therapies:
   [Patient] continues to worsen despite current [treatments]
   [Patient] has had an inadequate response to [treatment]
   [Patient] is unable to tolerate [treatment]
   [Treatment] is contraindicated for this [patient]
Documents diagnosis of specific condition are a fusion of initial statements, patient information, lab results, and physician's feedback:
   Date of original diagnosis
   Severity (Grade/stage 1, 2, 3, 4, etc.)
Relief for symptoms of specific condition to restore level of functioning exhibited prior to the onset of illness:
   Supports level and intensity of care, e.g., hospitalization and specific procedure or specific drug therapy indicated for specific condition.
Consequences of inadequate treatment 211 are a fusion of the reiterative restraining optimization process including data from proprietary initial statements, patient information, lab results, professional guidelines, and physician's feedback:
   Clinical consequences
      Increase complications due to inadequate treatment (e.g., wound healing decrease).
      Decreased mental acuity (e.g., increased confusion or ability to concentrate).
      Premature death (e.g., MI or stroke).
      Worsening of comorbid conditions (e.g., increase shortness of breath).
      Increased inflammatory response (e.g., symptom management is inadequate to match inflammatory response).
      Disease state progression (e.g., decrease in blood pressure, but not enough to be considered controlled).
      Side effects of previous treatments (e.g., facial swelling or cough).
      Reduce mobility or increase risk of falling (e.g., use of antipsychotics off label for movement disorders).
      Physical Consequences.
         Reduced mobility, requires oxygen therapy, loss of vision, etc.
      Economic Consequences
         Increased hospitalization (e.g., uncontrolled glucose levels (too high or too low).
         Increased emergency room visit (e.g., increase chest pain, or episode of syncope)
         Unable to continue working (e.g., uncontrolled movements prevent work).
         Disability due to inadequate treatment (e.g., uncontrolled movements required disability support).
         Increased likelihood of need for surgical intervention.
      Management Consequences (Infection requiring additional specialists and medications, increased fall risk, etc.).
      Medication compliance/Adherence Consequences (Taking meds 1 vs 4 times a day has dramatically different result. Forgetting to take epilepsy meds may cause more seizures resulting in further costs.)
      Behavioral/Mental Health Consequences (Addiction, suicide, depression, anxiety, etc.)
Patient Treatment Relevance 212
   Required specific treatment protocol based on life circumstances, e.g., young adult in school would benefit from longer dosing interval such as a once-daily medication.
   Intolerance, side effects, or allergy to preferred treatments.
Generally accepted standards of care.
   Supported by national specialist society guidelines for treatment, e.g., American Society of Rheumatology, American Medical Association.
   Key opinion leaders' consensus positions or white paper.
   Prestigious Medical School, e.g., Mount Sinai or Harvard Medical School.
   Specialists and/or network specialists standards.

As indicated above, these statements are synthesized by the system into a fusion of restrained data based upon the targets identified by the method. The original sources include multiple and various databases which are stored and sifted for targets. Examples include:
International al Classification of Diseases, Tenth Revision, Clinical Modification System Database
   ICD-10-CM Codes—Classifications, diagnosis codes representing conditions and diseases, related health problems, abnormal findings, signs and symptoms, injuries, and external causes of injuries
FDA Label (Drugs.com)
HCPCS Coding database
   Level I, II, III
   CPT coding database
AMA guidelines
Insurance Company Database (Medical and pharmacy coverage policy)
Specialty society guidelines (NCCN, ACR, etc.)
   Relevant tests to particular disease
FDA LABEL Database
   The drug labels and other drug-specific information on this Web site represent the most recent drug listing information companies have submitted to the Food and Drug Administration (FDA). (See 21 CFR part 207.)
      Recent major changes
      Indications and usage
      Dosage and administration
         First treatment cycle
         Subsequent treatment cycles
         Dosage adjustment based on hematology, laboratory values
         Dosage adjustment based on serum electrolytes and renal toxicity
      Dosage forms and strengths
      Administration
      Contraindications
      Warnings and precautions
      Adverse reactions
      Use in specific populations
      Overdosage
      Clinical pharmacology
      Nonclinical toxicology
      Clinical studies
      References
      Supply, storage, and handling, implications
      Patient counseling information
Agency for healthcare research and quality (AHRQ.gov)
Hayes manual.
HEDIS and NCQA Guidelines
National institute of health (NIH.gov).
Blue Cross Blue Shield Association Guidelines
Clinical Practice Guidelines (NCCIH Guidelines)

The method synthesizes the final statements by targeting the key factors identified. Patient specific information is then restrained and combined during the optimizing process, to synthesize tailored rationales which are specific to this patient. Other categories of data may also be obtained from the specific tests performed including: CT scan, urinalysis, coloscopy, lumbar puncture, renal toxicity, magnetic resonance imaging, ultrasonography, biopsy, specific variables, values, tests results, blood pressure, etc., and yet other categories can also include details of previous treatments and specific conditions.

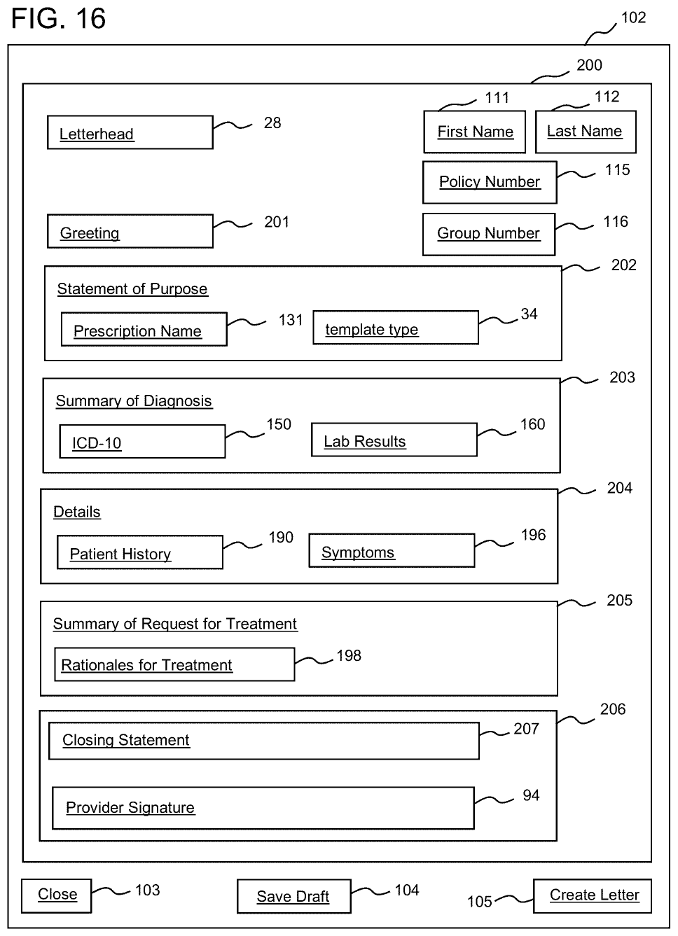

### FIG. 16—Viewing Final Draft 102

FIG. 16 is a diagrammatic view of the final draft page 102 of the program of the embodiment shown in FIG. 1. Following completion of the rationale summary page 198, the final letter preview 102 will appear on-screen. As this is a final draft 102, the user now has the additional options of saving the draft 104, closing the draft 103 to return to include yet further details, or to finalize and create the letter 105. If they save the draft 104, the letter will be named and saved in the archive 40. As previously discussed, all saved drafts 100 can also be accessed through the links along the header on most pages, or a specific draft can be accessed via the review draft button 101. Once a letter 200 is saved in the archive 40, the user can select that letter 200 to use as an additional template 35, 36, 37 for future letters. In streamlined embodiments, once complete, the letter will simply open in Acrobat for saving, printing, converting and/or sending. These embodiments may then be saved by the user.

As shown in FIG. 16, the final draft view page 102 shows a print preview of document 200. The patient information is shown in a designated portion of the document 200 according to specific medicament policy formats and may include data such as first name 111, last name 112, name of policy holder 113, policy holder DOB 114, policy number 115, and policy group number 116. This is retrieved from the patient information as shown in FIG. 10. A standard greeting 201 is provided, followed by a statement of purpose 202 including the prescription name 131 and the template type 34, i.e, a letter of medical necessity 35, a letter of appeal 36, or a letter requesting a formulary or medical exception 37.

The next section is a summary of the diagnosis 203 including details from the patient diagnosis 203 as discussed in reference to FIG. 13 and will include relevant details from the patient diagnosis 203, such as but not limited to, the ICD-10 150 and lab results 160. The next section thereafter according to some templates will be the further details 204 including the patient history 190 and patient symptoms 196 that support the purpose 202 of the present letter 200. The summary of rationale for treatment section 205 will include rationale for treatment statements from the tips section 60 and free-form rationales 199 entered and selected previously.

The final section 206 typically includes the standard closing statement 207 and provider information such as the provider first name 91, provider last name 92, provider title 93, provider practice name 94, provider practice address 95, and provider practice phone 96.

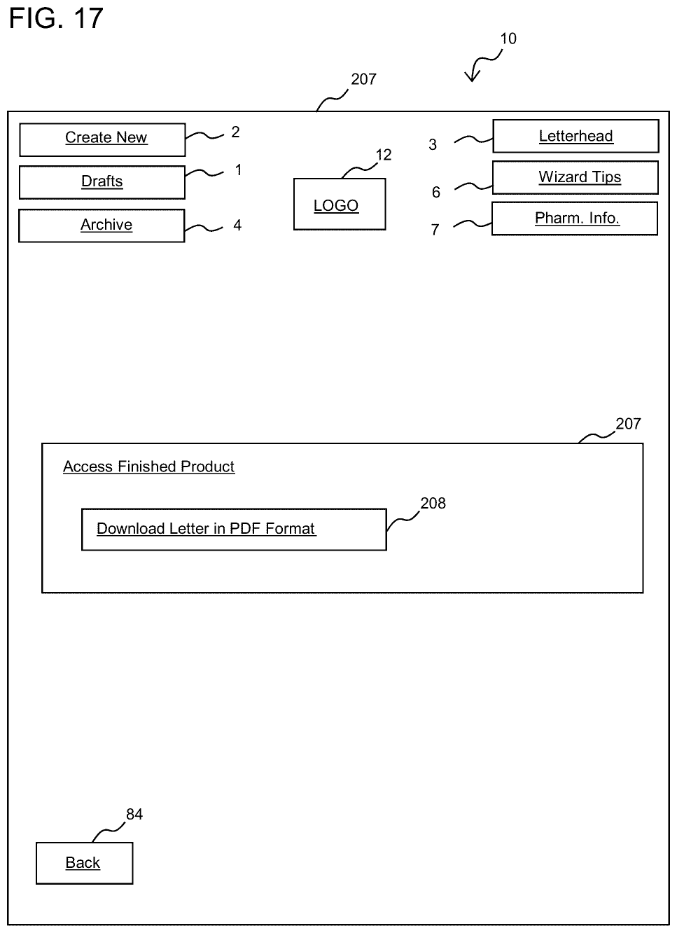

### FIG. 17—Download Final Product 207

FIG. 17 is a diagrammatic view of the final download product page 207 of the program of the embodiment shown in FIG. 1. Specifically, now that the final draft 102 has been reviewed, saved 104, and converted 105, it is available to access either directly here or from the document archive 40. Document 200 may now be accessed 207 and downloaded in pdf format 208. In other embodiments, an option to export directly to an outside communications portal (via email provider) may also be provided.

In one embodiment, if a user chooses to create the letter 105, they will be brought to a final product screen 207 asking them to click a button to download the letter in PDF format 208. In this embodiment, the letter 200 will then be downloaded locally to their computer as a PDF at which time instructions will be given on-screen about how to convert the PDF to a MS Word document.

In other embodiments, once a user chooses to create the letter 105, they will be brought to a final product screen 207 asking them if the product is complete. Once the user has confirmed that the letter is complete, the letter will simply open in Acrobat or other PDF program for saving, printing, converting and/or sending the letter in PDF format 208.

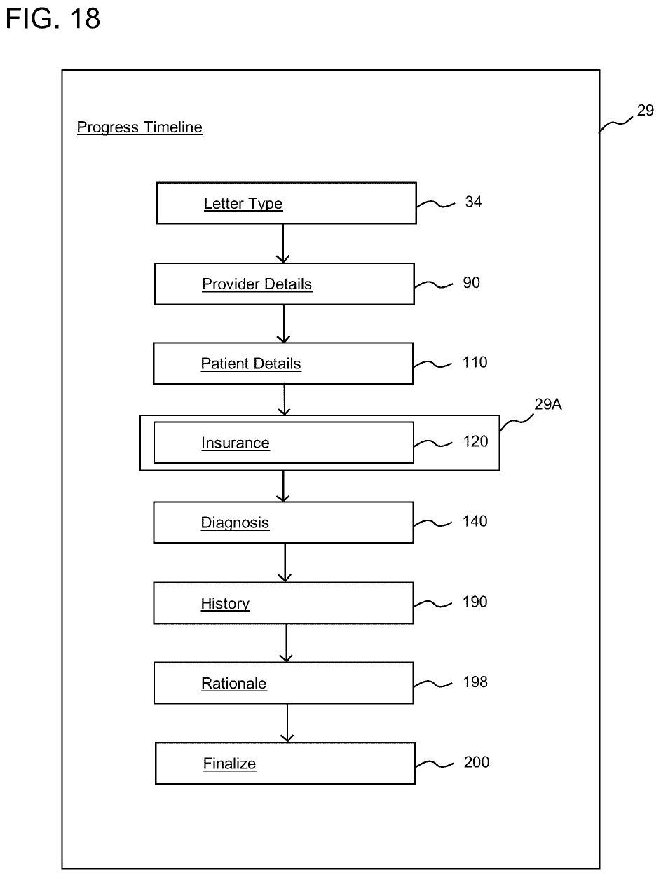

### FIG. 18—Method 250

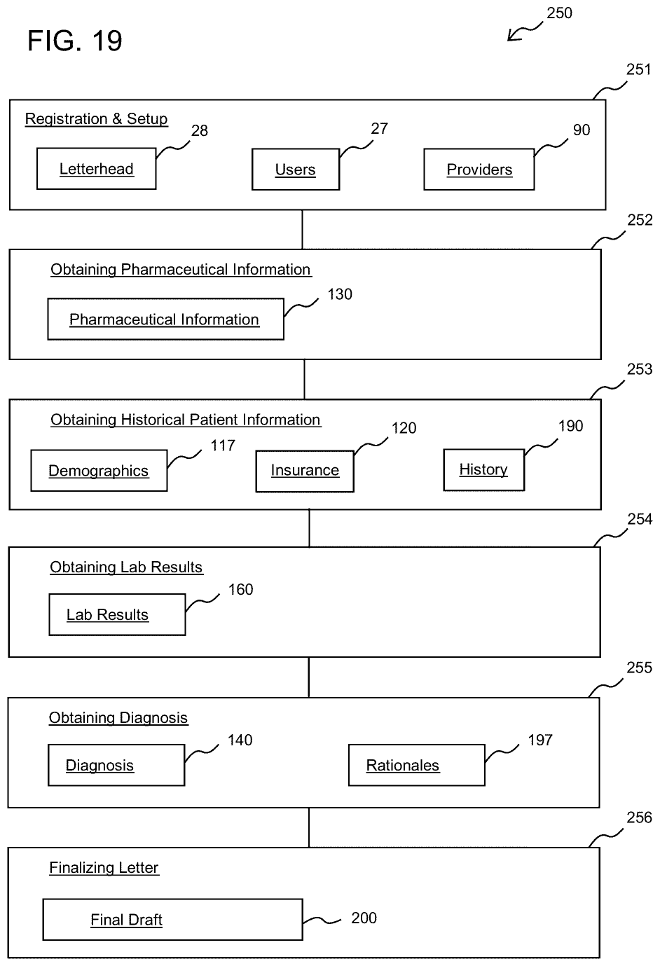

A diagram of the method steps 250 of the use of the present system 260 is provided in FIG. 19. Initially during registration and setup 251, the office administrator enters the details regarding the organizations' letterhead 28, users 27, and providers 90. The system provides pharmaceutical information 252 which was obtained by incorporating pharmaceutical information 130 from databases, again by office administrators. The system 260 facilitates obtaining all historical patient information 253 including patient demographic information 117, insurance information 120, and patient history 190 automatically through querying electronic health record platforms. The system facilitates obtaining relevant lab results 254 by gathering those lab results 160 from tests or procedures ordered by the doctor previously, may also be incorporated into the patient file by the office admin.

The only step requiring direct physician time is providing the rationales 255. Even here, the present system 260 expedites even this part of the process by providing writing tips 60 to facilitate expedient drafting overall. According to the present inventive method 250 then, the present method provides a system to address the problems that have been noted with prior art methods. The present system enables and expedites 1) addressing payer restrictions; 2) justifying treatments; 3) reducing letter writing hassle; 4) physician involvement time; 5) reduces complexity of letter writing; 6) increasing responsible delegation to staff; 7) automating data input; 8) increases completeness of letter content 9) providing specified overall training by user; 10) providing specific overall implementation protocols; 11) overall letter creation; 12) letter utilization; and 13) letter library management.

### FIG. 19—Progress Timeline 29

An example of the progress timeline is provided in FIG. 18. In order to provide a visual context for the user, a visual indicator, a progress timeline 29, is provided during the creation of the product 200. The main steps of creating the product: choosing the letter type 34, provider details 90, patient details 110, insurance information 120, diagnosis details 140, patient history 190, and physician rationales 198 are given visual indicators along the timeline 29. As the user progresses through the creation of the letter 200, a visual indicator 29A highlights or otherwise indicates which of the steps the user is on. As shown in FIG. 17, the visual indicator 29A is indicating that the user is currently importing the insurance 120 details.

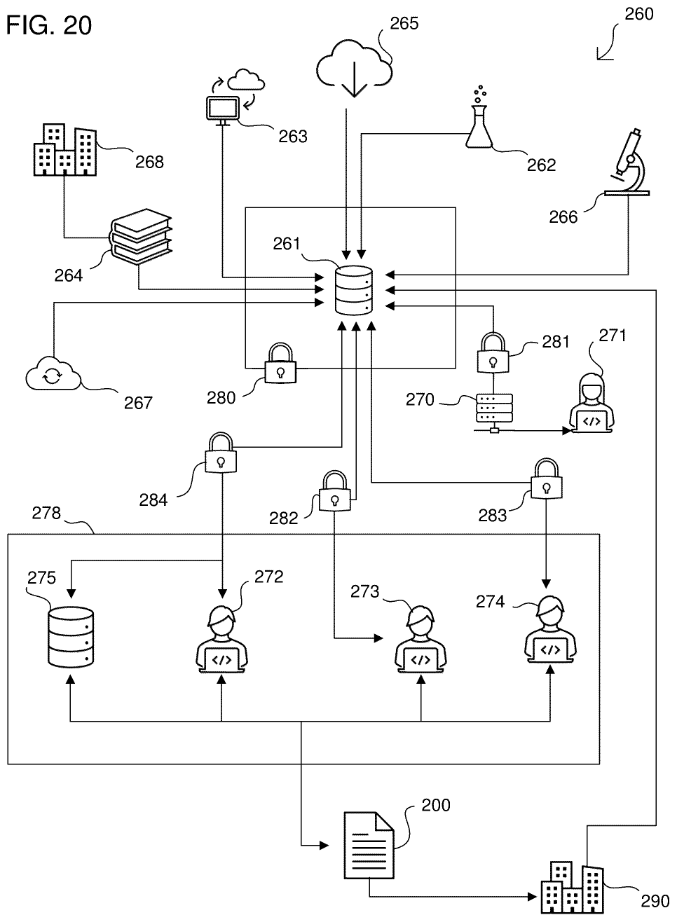

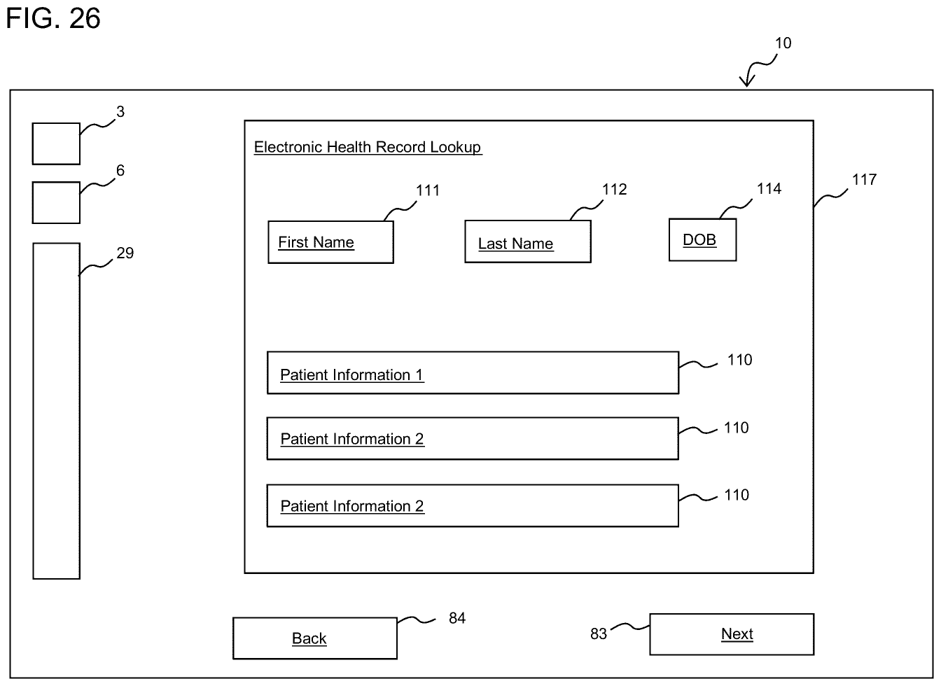

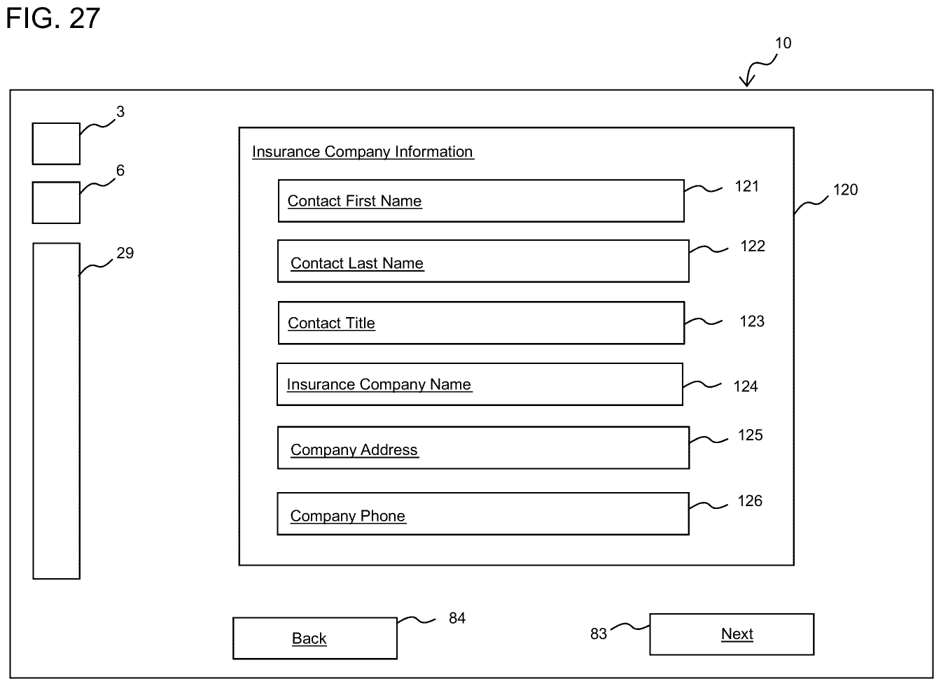

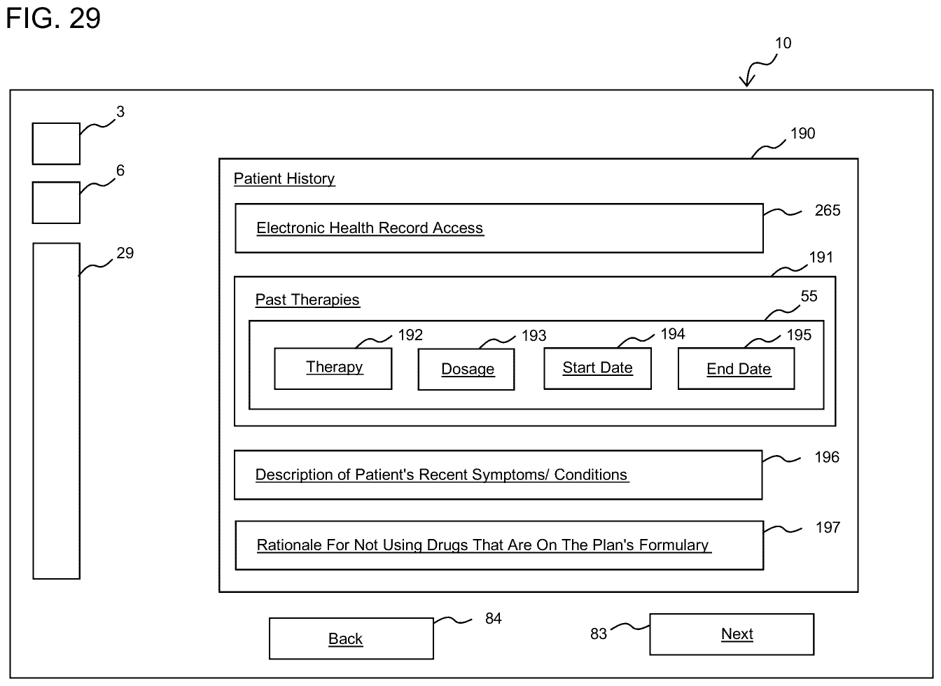

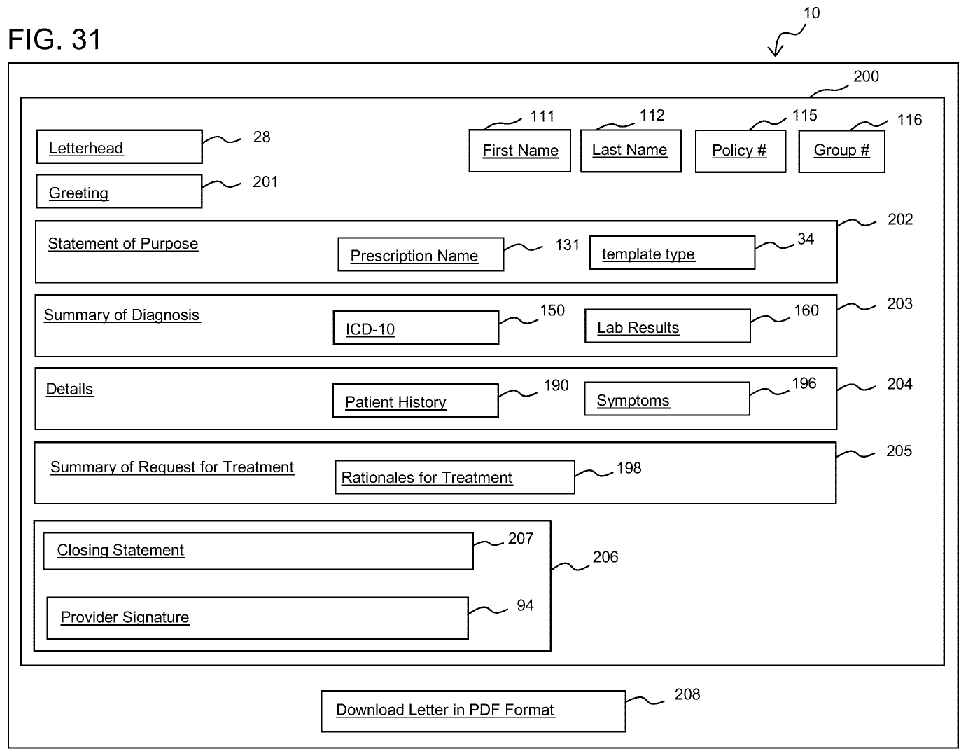

### FIGS. 20-32 Rheumatoid Arthritis Treatment Example

The following discussion of the system 260 is provided with reference to a hypothetical pharmaceutical and hypothetical patient profile which are provided for context so as to better understand the application of the method and the system of the present invention. Specifically, this illustrates the amount of factors which are made readily available by the system 260 for automatic letter creation after being retrieved and compiled from each of the sources 262, 263, 264, 265, 266, 267, 268 as discussed with reference to FIG. 20.

In this hypothetical case, the physician is filing an appeal so that the patient may have access to the representative pharmaceutical, windogo, which is in this case, being prescribed for rheumatoid arthritis.

In this example, the program is being funded by the pharmaceutical company 268 and provided for free, for physicians, for use with their pharmaceutical products, windogo being among them.

### FIG. 20—System 260

FIG. 20 provides a general overview of the system 260 according to one embodiment of the present invention. The program, letter drafts, and other data is stored on a HIPAA-compliant server 261 which can receive information from third party sources and systems.

While external systems and databases may, and are likely to, have their own security protocols, focus is directed towards the security system 280 which is part of the current system 260. Any outgoing data is protected by security 280, 281, 282, 283, 284. Specifically, the security measures 280, 281, 282, 283, 284 ensure that the information transfer to non-authenticated parties is strictly one-way. Thus, although the system 260 access in this example is funded by the pharmaceutical company 268, the patient information 110 remains secure, private, and separate from access by the pharmaceutical company 268. A dedicated on-call security agency 270 provides customized service portals and security interfaces 271 to provide on-call service, constant monitoring, and reliable security 281.

The security 280 may include both hardware and software components facilitating access control, transmission security, authentication, disposal, and encryption/decryption protocols. The system may provide security with access control to ensure that any software or application that stores patient health information restricts who can modify or access that data. The system may provide transmission security to safeguard the patient health information that is transferred over the communications network and between various parties through the system. The system may provide entity authentication to ensure that the entity accessing patient health information is the entity they are claiming to be. The system may provide permanent patient health information disposal where relevant to ensure patient health information security. The system may provide decryption/encryption to guarantee patient health information integrity. The system may provide data storage and backup as are necessary for data integrity. The system may provide automatic logoff after a specific period of inactivity. The system may provide single-sign-on (SSO) to enable users to gain access to an ecosystem of apps and sites quickly and efficiently without sacrificing privacy. The present system overview 260 shows integration via secure communications protocols with a few exemplary third-party sources/databases. It is to be understood that these are just a few examples of the databases/external sources which are culled for the necessary data. Other databases and external sources which are employed include, but are not limited to: International Classification of Diseases, Tenth Revision, Clinical Modification System Database, ICD-10-CM, FDA Label (Drugs.com), Current Procedural Terminology (CPT)/Healthcare Common Procedure Coding System coding database, Level I, II, III coding database, American Medical Association guidelines, the relevant insurance company databases (medical and pharmacy coverage policies), specialty society guidelines (Council of Medical Specialty Societies, American Academy of Allergy, Asthma & Immunology, American Academy of Dermatology, American Academy of Family Physicians, National Comprehensive Cancer Network, American College of Radiology, etc.), Food and Drug Association FDALABEL Database, Agency for Healthcare Research and Quality database (AHRQ.gov), Hayes Manual, National Committee for Quality Assurance' Healthcare Effectiveness Data and Information Set guidelines, National Institute of Health (NIH.gov), Blue Cross Blue Shield Association guidelines, and the clinical practice guidelines from the National Center for Complementary and Integrative Health.

Each of these third-party databases are a source of external data relevant to their specific field which may be automatically imported by the present system 260 to facilitate and expedite letter creation. Pharmaceutical information 70 is obtained from pharmaceutical companies 268 and other pharmaceutical databases 264. Patient information 110 and patient history 190 is imported from electronic health record platforms 265. Patient history 190 may also be obtained from previously used laboratories 266. Recent test results 160 may be obtained from different laboratories 266 if other laboratories 262 were used previously.

Within the physician's office 278, just as admins and nurses have their own respective interfaces 274, 273, physicians also have their own interfaces 272. If letter drafts are enabled to be saved onto the secure database 261, admins and nurses can save drafts which may be accessed onto that database 261. Alternatively, admins and nurses can save incomplete letters onto the physician's office database 275. This enables the admins and nurses to reduce the associated physician's workload while still ensuring that physicians have the final say over the final letter 200 before it is sent to the insurer or insurance agency 290.

It is to be understood that while the system shown in FIG. 20 illustrates a single server, this may in fact represent a plurality of servers, a plurality of networked computing devices, or other computer workstations. Regardless of the server type that is employed, the system will be associated with a communications interface, memory, security hardware, and processing circuitry which may be in communication with one another via a bus. The communications interface will be further in communication with a secure communications network facilitating secure communication between the server, security platform, pharmaceutical company, a plurality of physician offices, a plurality of third-party sources/databases (as listed above), the insurance company, the laboratories, and the electronic health record platforms. The security hardware will be capable of supporting security features such as SSL certificates and may be a security chip, such as a trusted platform module (TPM) version 2.0.

The memory may include one or more types of memory such as main memory, primary memory, nonvolatile memory, read-only memory, programmable memory, electrically erasable programmable read-only memory, volatile memory, flash memory, or other nonvolatile memory storage medium that can be reprogrammed and electrically erased. Specifically, memory may be a physical component either external or internal to a main device capable of storing information while the system is powered on or retaining information even when the system is powered off. The memory is capable of being coded to store information, database copies, a series of on bits and off bits, data, content, contact lists, database lists, wizard tip lists, applications, programs, instructions, protocols, category targets, and method processes. Volatile memory and nonvolatile memory may have differing respective speeds. Together the memory enables the system to operate according to the present inventive methods disclosed herein. For example, the memory may be configured to buffer patient bibliographic data 111, 112 for processing with the connected EHR system, further configured to store pharmaceutical information 70 for later access by physician offices 278, and routinely configured to store directives for execution by the processing circuitry including processes according to the present restraining method and their respective variables including each of: inquiries, decisions, and variable outputs.

The processing circuitry may include one or more processors working in tandem or independent of one another. The hardware involved may include digital logic circuits, circuit boards, combinational logic circuits, Boolean circuits, analog circuits, op amp circuits, analog integrated circuits, microprocessors, integrated processors, integrated analog circuits, integrated digital circuits, and mixed-signal integrated circuits. These processors may contain circuits with logical function and connectivity which may be programmable by the user for various functions including logic gates, adders, and registers. These processors may be codified to fetch instructions from storage; interpret the fetched instructions; process data communicated via bus according to other instructions both from memory and storage; allocate commands for other server components; and send output instructions and data to storage according to the fetched instructions and commands.

The communication interface may be any of several types of hardware, wired or wireless, being capable of receiving and transmitting data from other devices via the communications network and capable of meeting the latency and bandwidth requirements according to the required parameters for transporting a large amount of data quickly.

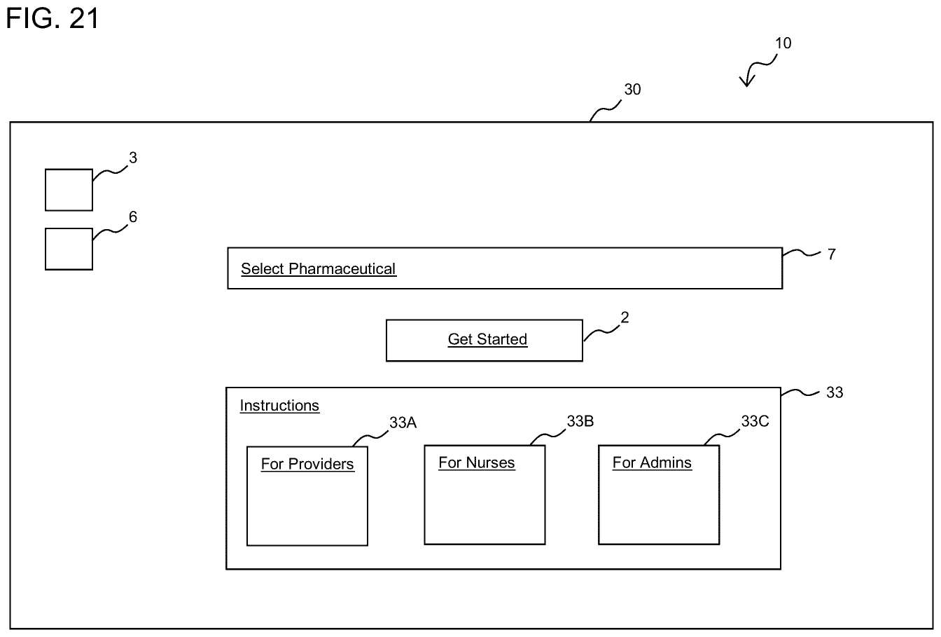

### FIG. 21—Starting 30

As shown in FIG. 21, in this embodiment, the system has a streamlined main starting option 30 in which the links may be accessible via a quick access drop-down menu which may be found on the top or top-left of the screen. The quick access menu may contain user profile information, home, letter preview, and start letter functionality. In one embodiment, the quick access menu is customizable so that it may be fully visible or collapsed to simplify the view according to the preferences of each user. The user can select the pharmaceutical product link 7 from a drop-down menu list for manufacturers with more than one product.

A quick start guide may be provided with introductory training video sessions with different instructions 33 depending upon the classification level of the user audience. Instructional videos may include instructions 33 on how to generate the first medical letter, how to provide access for office staff for nurses, and specifically, for office admins, on how to get the entire office set up if not already previously done so.

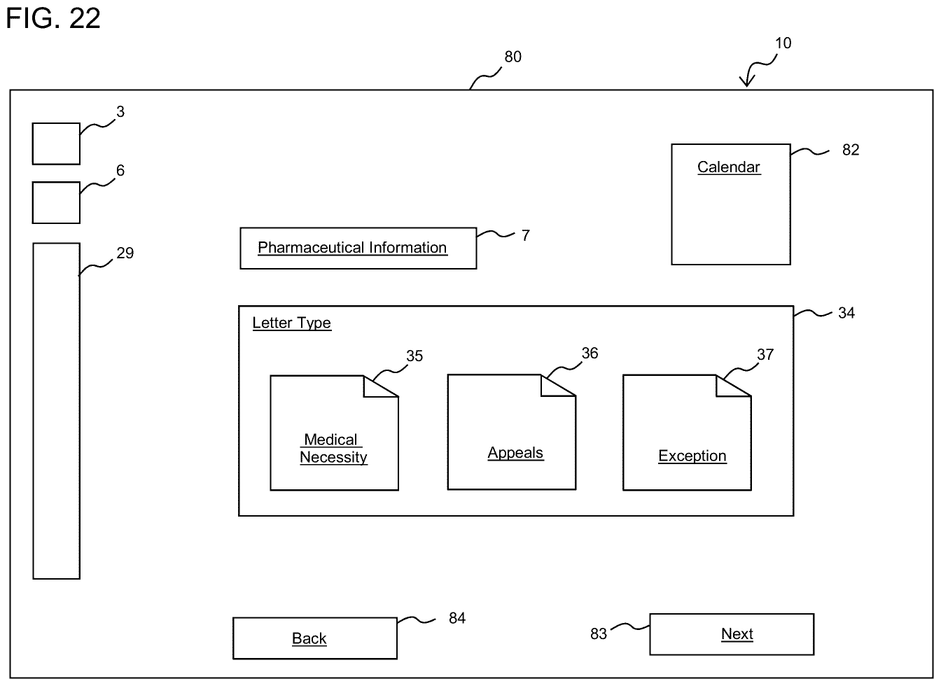

### FIG. 22—Creating New Letter 30

In this example, the office admin 274 watched the instructions for admins 33c and has already set the system 260 up for future use. They imported company information including the addition of a logo, company/practice information such as company name, address, phone, and fax. The office admin has likewise imported the insurance company information.

As part of the optimization process to reduce the associated physician time, the admin may access the starting page 30 of the present invention. In this situation the admin chooses the appeals template from among the available template types 34 since this is a letter appealing the decision to deny access for windogo. The system continues to provide prompts for the admin to continue to enter and confirm the data suggested by the present method. Once the system has determined that the admin has entered or confirmed all of the data appropriate to the admin classification level, the system prompts the admin to save the letter draft after completing those portions of the letter. The system prompts the nurse to access the data and begin to enter or confirm all of the data appropriate to the nurse classification level. Once the system has determined that the nurse has entered and confirmed all of the data appropriate to the nurse classification level, the system prompts the physician to enter or confirm all of those details which can only be entered and confirmed by the physician.

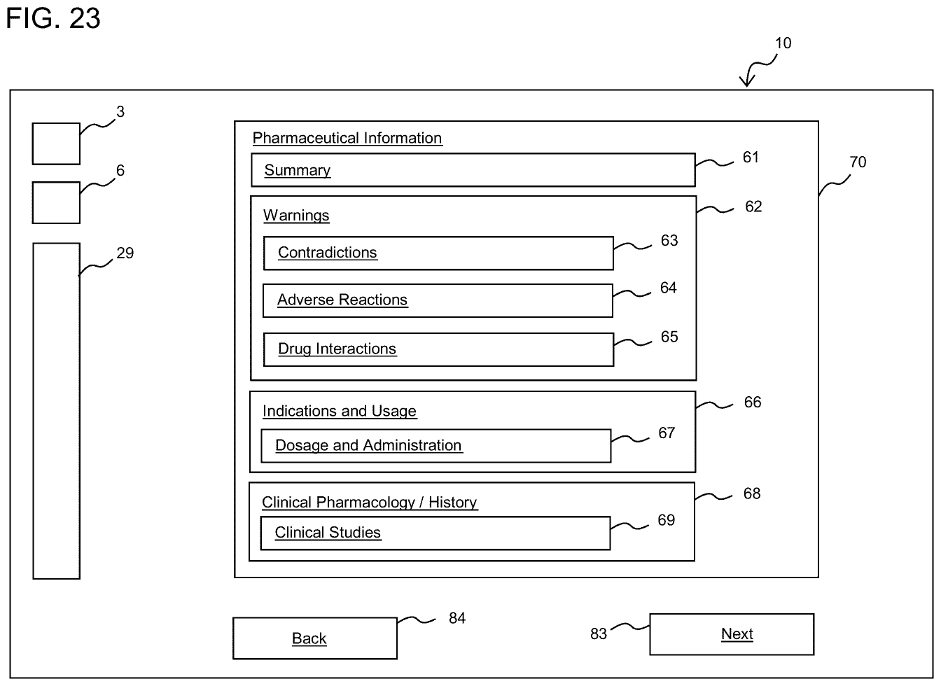

### FIG. 23—Pharmaceutical Information 70

The pharmaceutical company 268 provides pharmaceutical information 70 from their pharmaceutical information database 264. This may include information found on the pharmaceutical manufacturer's package information data sheet and other data. The pharmaceutical information 70, see FIG. 7, would then include at least the highlights of the available prescribing information.

The pharmaceutical information 70 for pharmaceutical “windogo” may include the following disclaimer: “These highlights do not include all the information needed to use windogo safely and effectively. See full prescribing information for windogo.”

The summary 71 for windogo may include details regarding the pharmaceutical including the type: “Windogo windogocapsules, tablets, or powder for oral suspension. Initial U.S. Approval: 1974.” The summary 71 may include a brief recitation of recent major changes including: “Those changes to indications and usage 76: gonorrhea (1.5) removed September 2015; and dosage and administration 77: gonorrhea (2.1) removed September 2015.”

On the pharmaceutical information page 70, the summary 71 may include a brief overview of indications and usage 76:
“Windogo is a penicillin-class antibacterial indicated for treatment of infections due to susceptible strains of designated microorganisms. Indicated for: infections of the ear, nose, throat, genitourinary tract, skin and skin structure, and lower respiratory tract. (1.1-1.4). Recommended in combination for treatment of h. Pylori infection and duodenal ulcer disease. (1.5).”

Summary 71 may include a brief overview of warnings 72. For example, “To reduce the development of drug-resistant bacteria and maintain the effectiveness of windogo and other antibacterial drugs, windogo should be used only to treat infections that are proven or strongly suspected to be caused by bacteria. (1.6).”

Summary 71 may include a brief overview of dosage and administration 77 according to use for specific populations, for example:
“In adults, 750-1750 mg/day in divided doses every 8-12 hours; in pediatric patients >3 months of age, 20-45 mg/kg/day in divided doses every 8-12 hours. Refer to full prescribing information for specific dosing regimens. (2.1, 2.2, 2.3). The upper dose for neonates and infants≤3 months is 30 mg/kg/day divided every 12 hours. (2.2); dosing for h. Pylori infection: triple therapy: 1 gram windogo, 500 mg clarithromycin, and 30 mg lansoprazole, all given twice daily (every 12 hours) for 14 days. Dual therapy: 1 gram windogo and 30 mg lansoprazole, each given three times daily (every 8 hours) for 14 days. (2.3). Reduce the dose in patients with severe renal impairment (gfr<30 ml/min). (2.4). Dosage forms and strengths according to dosage and administration 77 are also listed, including: capsules: 250 mg, 500 mg (3); tablets: 500 mg, 875 mg (3); powder for oral suspension: 125 mg/5 ml, 200 mg/5 ml, 250 mg/5 ml, 400 mg/5 ml (3).”

Summary 71 may include a brief overview of the warnings 72 which includes contradictions 73, adverse reactions 74, and drug interactions 75, for example:
“Contraindications 73 for this pharmaceutical include a history of a serious hypersensitivity reaction (e.g., anaphylaxis or stevens-johnson syndrome) to windogo or to other beta-lactams (e.g., penicillins or cephalosporins) (4). Warnings and precautions 72 for this pharmaceutical include anaphylactic reactions: serious and occasionally fatal; anaphylactic reactions have been reported in patients on penicillin therapy. Serious anaphylactic reactions require immediate emergency treatment with supportive measures. (5.1) Further warnings include Clostridium difficile-associated diarrhea (ranging from mild diarrhea to fatal colitis): evaluate if diarrhea occurs. (5.2). Adverse reactions 74 for this drug include the most common adverse reactions (>1%) observed in clinical trials of windogo capsules, tablets or oral suspension which were diarrhea, rash, vomiting, and nausea. (6.1). The warnings also providing a reporting protocol statement: “To report suspected adverse reactions, contact Dr. Teddy's laboratories inc., at 1-888-375-3784 or fda at 1-800-Fda-1088 or www.fda.gov/medwatch.”

The warnings 72 may also include a list of drug interactions 75 including, for example:
“Probenicid decreases renal tubular secretion of windogo which may result in increased blood levels of windogo. (7.1). Concomitant use of windogo and oral anticoagulants may increase the prolongation of prothrombin time. (7.2). Coadministration with allopurinol increases the risk of rash. (7.3). Windogo may reduce the efficacy of oral contraceptives. (7.4). The warnings also include a warning for use in specific populations, i.e, pediatric: modify dose in patients 12 weeks or younger (≤3 months). (8.4).

Summary 71 may include a brief overview of the warnings 72 which includes contradictions 73, adverse reactions 74, and drug interactions 75. Indications and usage 76 may include dosage and administration 77 according use for specific populations. Clinical pharmacology and history 78 may further include additional details such as any clinical studies 79 which were relevant. The full prescribing information 70 may continue to be provided for each of the above sections in further and greater detail.

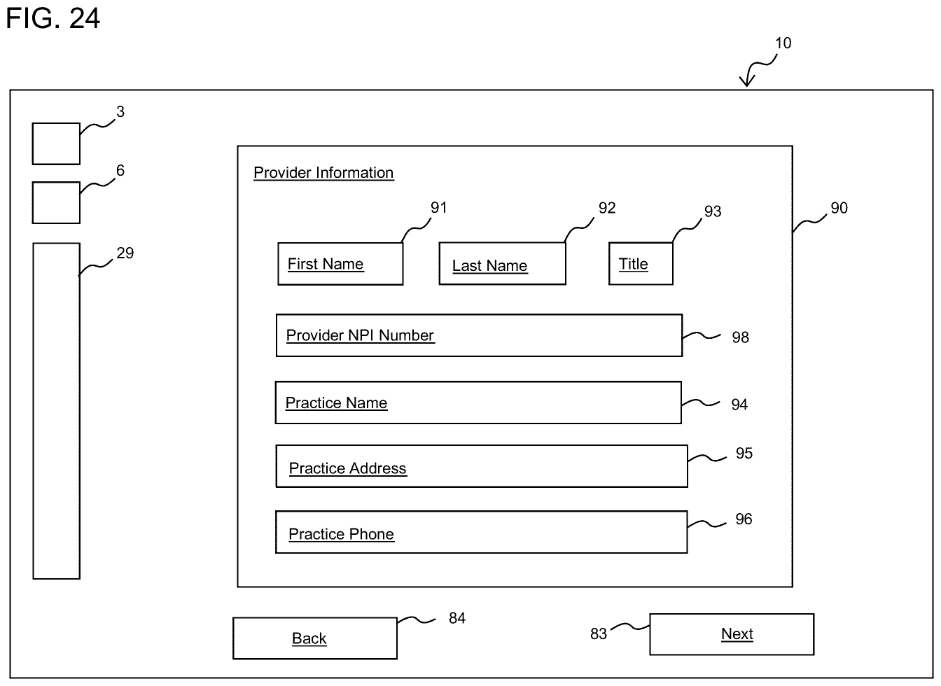

### FIG. 24—Provider Information 90

Again, as previously stated, in this example, the office admin 274 watched the instructions for admins 33c and has already set the system 260 up for future use by this practice. The admin already enabled the system 260 to automatically import the provider's company information, including logo, practice name 94, address 95, phone 96, and fax from the contact data of the physician's private network and database 275. The admin also enabled the system 260 to automatically import the provider's information 90, including name 91, 92, 93, and NPI number 98, from contact lists in the physician's private network 275. The physician only needs to review this information 90 in order to ascertain that the right prescribing physician's name is associated with the correct patient and letter 200.

Again, in other embodiments, it is possible that the contacts may be obtained from another database and may be offered as alternative contacts in a drop-down formation, so that the system may still facilitate automatic importing into the present system 260, and expedited datafill.

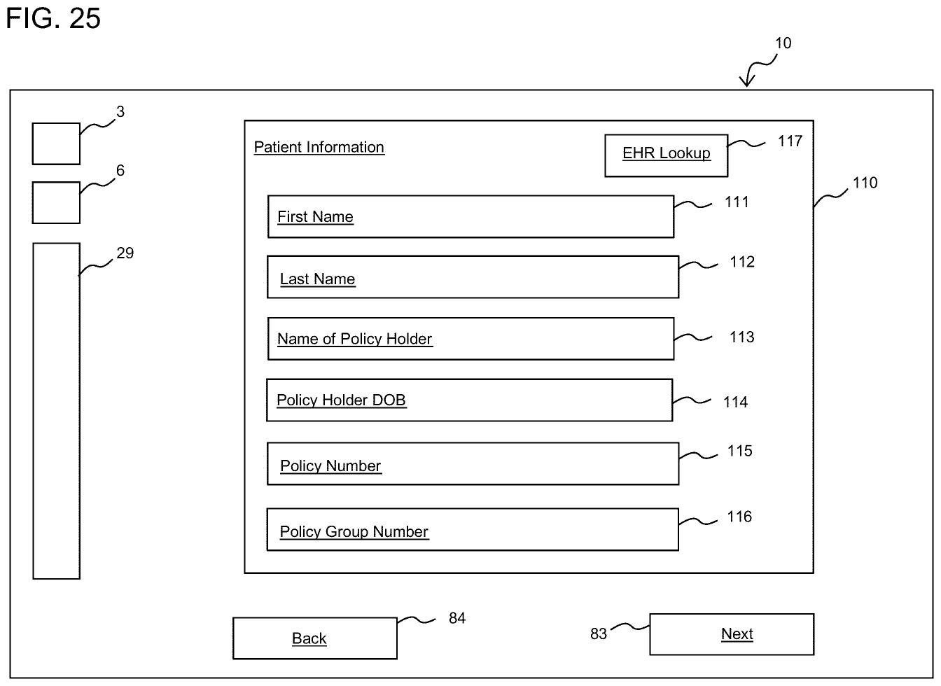

### FIGS. 25-29—Patient Information 110

The patient information 110 is imported from the electronic health record platforms 265. A further toggle button facilitates duplicating the information into the patient insurance policy holder text boxes 113, 114, 115, 116. At times, this access is automatic, at other times, a separate button 117 along the top of the interface serves both as a reminder and to facilitate allowing the user to click to connect the system 260 with the electronic health record platform, such as EPIC. In one embodiment, a pup-up window will display the electronic health record platform access page allowing the admin user to enter details such as the patient name 111 and date of birth 114 to search for the full patient records. Search results will propagate with further details 110 allowing the user to confirm that the right patient has been identified.

The admin identifies and confirms that according to the patient information 110 profile, the patient is a 47-year-old female diagnosed with rheumatoid arthritis (RA) 10 years ago. There is a patient history 190 of bilateral inflammation of the knees and hands 196. Positive rheumatoid factor and elevated CRP>10 mg/L 160. Patient complains of morning stiffness >45 minutes and difficulty ambulating >15 minutes 170. Patient was initially prescribed methotrexate 192 and steroids 192 as needed with good relief but over a period of 9 months 194, 195 symptoms returned and did not respond to increased dosing of MTX 193. Steroids were still effective but used sparingly due to concerns of long-term side effects 197. In future embodiment, the information would selected from patient chart based in the input of a single variable such as the identification of the indication using the ICD10 database.

Imaging 160 indicated significant joint space narrowing and progression of joint space narrowing in both knees and MCP joints in both hands. Patient was prescribed Windogo® but was denied access by her insurance company.

Upon further investigation the health plan pharmacy policy required a minimum of 12 months on methotrexate before any biologic agents would be approved, and preferred agents must be tried and proven ineffective or that the patient is not able to tolerate the drug at a clinically effective dose. In addition, the patient must have a confirmed diagnosis of RA, be over the age of 18, and have no ongoing infections, including TB for which they must be tested in advance. The hypothetical available biologics and formulary/coverage status is: windogo: non-preferred; Remicade: non-preferred; enbrel: non-formulary; cimzia: preferred brand specialty tier; cosentyx: non-preferred; rituximab: non-preferred; tremfaya: formulary exclusion; and stelara: preferred brand, specialty tier.

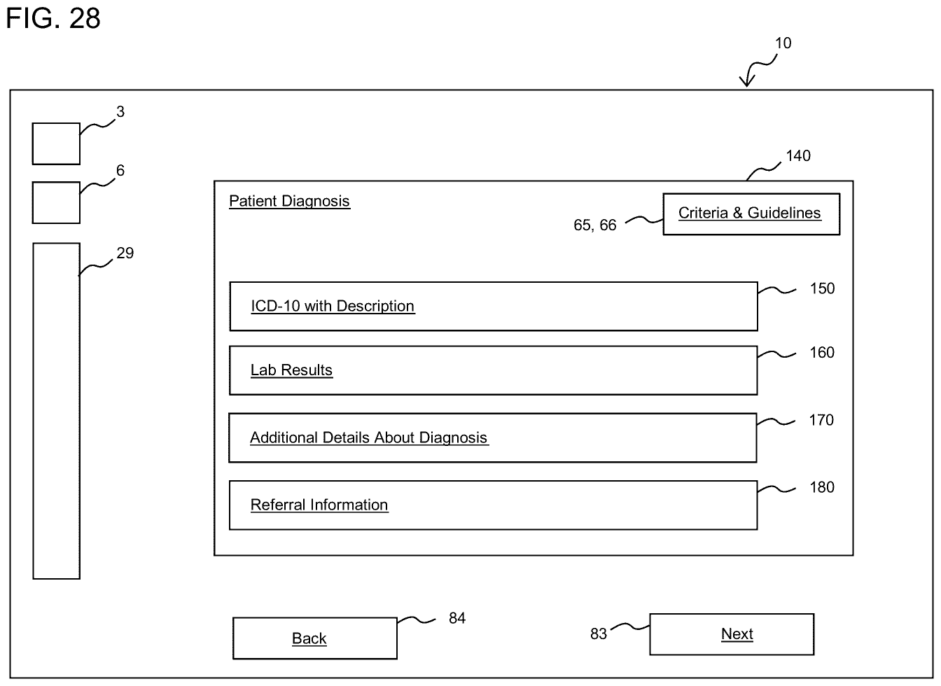

When accessing the patient diagnosis 140, see FIG. 28, the various relevant diagnosis of specific conditions are obtained and provided as possible options from the ICD-10 CM database 150 in dropdown format:
M05 Rheumatoid arthritis with rheumatoid factor
M05.9 Felty's syndrome, multiple sites
M06.00 Other rheumatoid arthritis
M06.011 Rheumatoid arthritis without rheumatoid factor, right shoulder.
M06.012 Rheumatoid arthritis without rheumatoid factor, left shoulder.
M06.019 Rheumatoid arthritis without rheumatoid factor, unspecified shoulder.
M06.021 Rheumatoid arthritis without rheumatoid factor, right elbow.
M06.022 Rheumatoid arthritis without rheumatoid factor, left elbow.
M06.029 Rheumatoid arthritis without rheumatoid factor, unspecified elbow.
M06.031 Rheumatoid arthritis without rheumatoid factor, right wrist
M06.062 Rheumatoid arthritis without rheumatoid factor, left knee
M06.9 Rheumatoid arthritis, unspecified
Date of original diagnosis (Dropdown date calendar)
Severity Grade
1
2
3
4

Again, by providing each relevant option as a dropdown option, the user may easily click through the screen, and quickly obtain the correct option. The system thereby greatly reducing the overall time spent in drafting this portion of the letter.

Lab and diagnostic test results 160 may be imported automatically from laboratories 262, 266 if authorized. However, not all laboratories enable automatic direct patient test result exportation.

To expedite letter creation in these situations, relevant diagnostic test result options are provided from the relevant professional society guidelines databases 276 in dropdown format with the following relevant options, each of which, when selected have further relevant options in dropdown menus, with the date of each test indicatable:
X-rays
   Left
   Right
   Hand
   Knee
   Hip
   Ankle
   Vertebrae
CT Scan (Dropdown)
   Left
   Right
   Hand
   Knee
   Hip
   Ankle
   Vertebrae
   Back
   Pelvis
CRP
   <10
   >10
Blood counts
   WBC
      4-10
      <4
      >10
   RBC (Dropdown)
      3.58-5.19
      >5.19
      <3.58
   HGB (Dropdown)
      11-15.5
      <11
      >15.5
   Rheumatoid Factor (Dropdown)
      <15 lu/ml
      >15 lu/ml

Again, by providing each relevant option as a dropdown option, the user may easily click through the screen, and quickly obtain the correct option. The system thereby greatly reduces the overall time spent in drafting this portion of the letter.

As discussed above, if obtained as part of the patient history, prior therapies 192 are obtained and imported from electronic health record platforms. However, there are times when this data cannot be automatically imported. The system 260 provides prior therapy options 192 for the user to pick and choose from quickly to eliminate time in typing up the past therapies all over again. This also ensures that the final letter 200 has only the past therapies listed that are relevant to the current diagnosis and not the full patient history (reducing the irrelevant information, preserving patient privacy, and reducing the size of the letter).

Prior therapies 192 options are generally displayed in dropdown format. These prior therapy options 192 include both drug therapies and non-pharmacological interventions. Relevant pharmacological options are obtained from the Health Plan Coverage and Pharmacy Policy databases, such as MMIT and Fingertip Formulary. Non-pharmacological interventions are obtained from the Health Plan Coverage and Pharmacy Policy databases, as well as professional society guideline databases.

Each category includes further relevant options, each of which, when selected have further relevant options in dropdown menus, with the date of each test indicatable:
Drug therapies, generics
   Methotrexate
   Sulfasalzine
   Steroids
   NSAIDS
Drug therapies, Brands
   Windogo
   Remicade
   Enbrel
   Cimzia
   Cosentyx
   Rituximab
   Tremfaya
   Stelara
Non-pharmacologic interventions
   Physical therapy
   Occupational therapy
   Medical or Surgical interventions
   Hyaluronic Acid injections
   Steroid Injections
   Joint replacements

For each of the prior therapy options 192, options are provided for the prescribed dosage 193 in dropdown format. Prescribed dosage types, frequency, and amounts are obtained from the FDA-labelled dosage, Health Plan Coverage and Pharmacy Policy databases and integrated according to the method of the present invention. Examples for the prescribed dosage 193 are provided below:
Types
   oral drug delivery
   intravenous (IV) infusion
   intramuscular injection
Amounts
   10 mg
   20 mg
   30 mg
   40 mg
   50 mg
Frequency
   1×/day
   2×/week
   2×/month
   1×/month

Again, by providing each relevant option as a dropdown option, the user may easily click through the screen, and quickly obtain the correct option. The system thereby greatly reduces the overall time spent in drafting this portion of the letter.

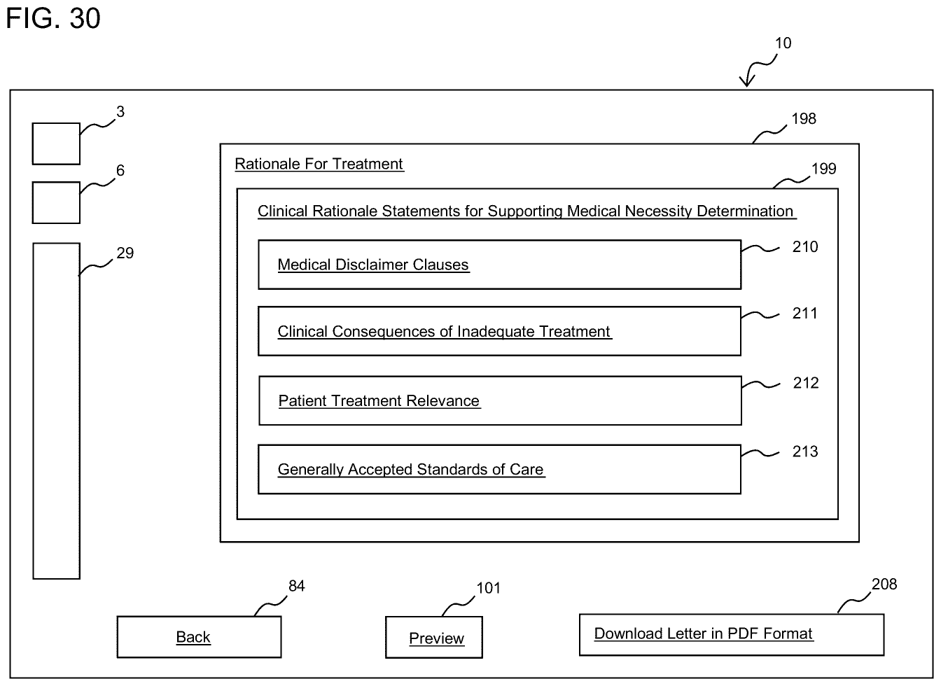

### FIGS. 30 & 31—Rationales & Final Letter

Multiple options for suggested relevant rationales 199 are provided for the treatment for rationales 198 in dropdown format. These rationales include a summary of the professional opinion of the patient's likely prognosis, risks of negative outcomes without treatment with windogo including contradictions or other limiting factors 210. Clinical consequences of inadequate treatment 211 are obtained and updated from professional society guidelines database. Generally accepted standards of care 213 are likewise obtained from professional society guidelines database. Examples are provided below:
Patient continues to show worsening joint degradation despite current treatments
Patient has had an inadequate response to current treatment with MTX
Patient is unable to tolerate MTX
Treatment is contraindicated for this patient
Clinical consequences of inadequate treatment
   Physical Consequences
   Reduced mobility
   Chronic Pain
   Joint replacement
   Joint injections
   Increased/additional pain medications/opioids
   Impact on comorbidities
   Increased fall risk
   Depression/anxiety
   Economic Consequences
   Increased likelihood of hospitalization
   Need for surgical intervention
   ER
   Doctor visits
   (Space for entering free text).
Generally accepted standards of care (Database: Professional society guidelines)
   ACR guidelines
   Published data
   Regional standards of care
   Key opinion leader support

Again, by providing each relevant option as a dropdown option, the user may easily click through the screen, and quickly obtain the correct option. The system thereby greatly reducing the overall time spent in drafting this portion of the letter.

As before, the user can review the compiled information included in the final letter 200 including the business letterhead 28, suggested greeting 201, patient information 110 (specifically bibliographic information 111, 112, insurance information 115, 116), statement of purpose 202, summary of diagnosis 203, further details 204, summary of request for treatment 205, and conclusion 206.

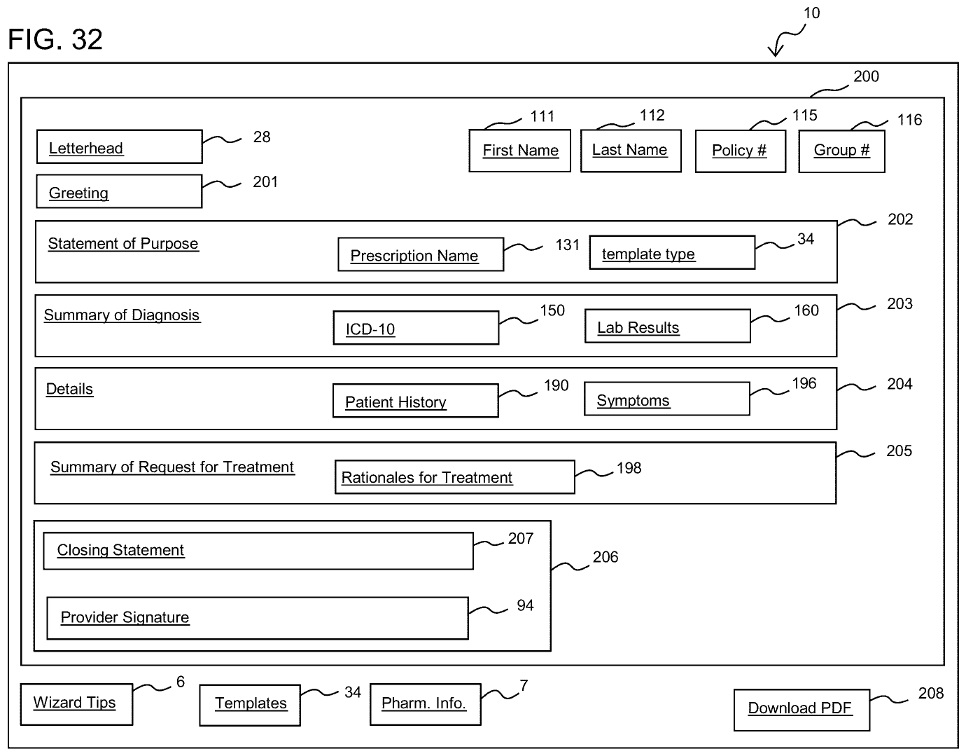

### FIG. 32—Single Screen Format

While the present display format discussed above has been to emphasize the different data imported one at a time for clarity, another embodiment of the present invention employs a one-page format for importing each aspect of the invention and seeing the letter updated in real time in the screen in front of the user. The present invention thereby provides a method not only of obtaining the required information from multiple sources, but also of ensuring increased oversight that only particularly relevant data is available for inclusion into the final letter product 200.

### Method

The present inventive method provides an access letter writing platform that simplifies and improves the letter writing process and content allowing providers and their staff to quickly develop effective patient-specific access letters. The present inventive method is a computer-implemented method enabled by a proprietary, secure web-based platform being hosted in a HIPAA and HITECH compliant environment, being secured by fully managed firewalls. Strict user controls merge seamlessly with continuous third-party monitoring for continuous ongoing security. Business rules and customer service resources are designed to support and manage office-level needs and technical support. Intuitive series of customized prompts result in a comprehensive and customized access letter regardless of where users start the program. Practice information (prescribers, practice logo, etc.) is saved during the account creation process to further simplify the letter writing process from previously available methods. Secure servers with security enabled authentication protocols enable letters to be e-signed by the provider to further expedite the creation process.

The present invention provides a method to achieve provider efficiency for the administrative process, enable customization for a specific prescribed pharmaceutical drug, enable placing the financial cost for upgrade on the specific select pharmaceutical manufacturer, and yet maintain compulsory patient confidentiality and required HIPAA compliance to the strictest degree.

The present system uses a secured communications network system, to provide a customizable letter template for numerous drugs and devices. This present invention further facilitates a method of tailoring a letter for a specific named patient, with a specific precise history, with specific unique symptoms, for a specific prescribed pharmaceutical, for a specific distinguishing purpose, with specific tailored tips to address concerns of the particular insurance company's s' restricted coverage programs.

As previously discussed, according to the US Census Bureau and other industry statistics within the United States, as of 2023, there are over 345 million patients, over 1.1 million active physicians, over 400,000 insurance brokers & agencies businesses, over 20,000 different prescription drug products, and approximately 14 billion laboratory tests are performed each year. The present inventive method facilitates creation of a letter of medical necessity, an appeal, or request for formulary exception for prescriptions for a patient based upon a unique combination of factors from potentially each of these sources. Where the number of unique combinations of m, n, o, p requirements with r categories is determined by the formula: C ((m,n,o,p),r)=n!(n−r)!+0!(o−r)!+m!(m−r)!+p!(p−r)!, which due to the numbers involved, results in an almost infinite number of possible combinations.

The present invention overcomes the difficulties of the present epidemic of infobesity and infinitely generated combinations by using a method of identifying targets within each of the relevant data categories to facilitate a method of reducing the options thereby tailoring a customized letter for a prescribed drug, with a specific appropriate utilization management criteria, with an established verification protocol with an exclusive optimization protocol, for a prescribed pharmaceutical, for a specific limited purpose, with specific tips to address concerns of the insurers' compliance programs.

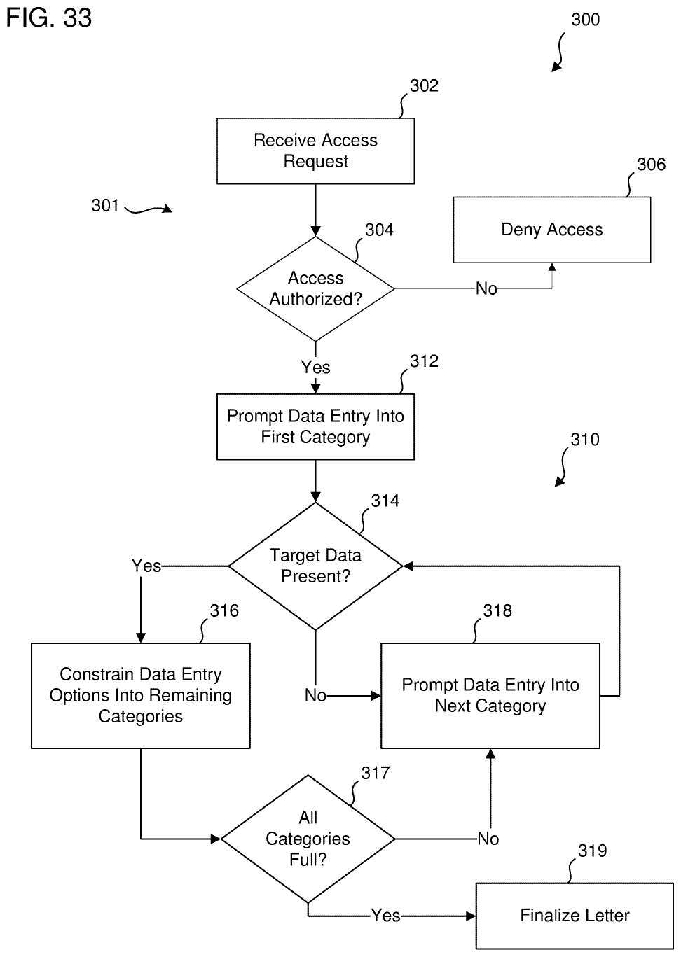

### FIG. 33—Optimization & Reiterative Restraint Process

The present invention provides a method not only of obtaining the required information from multiple sources, but also of ensuring that only particularly relevant data is available for inclusion into the final letter product. To facilitate this, targets are identified within each category provided by third parties. These identified targets are reiteratively utilized to further restrain the results within the other categories.

FIG. 33 shows that a user is attempting to access patient information 110. The inventive optimization method 300 employed in the rheumatoid arthritis example above from the system's perspective starts when the system receives a request to access 302 patient information. Due to the security hardware and present, the system will prevent any access that is not authorized, so the processor analyzes whether access is authorized or not 304. If authorization is not authorized, the system will deny the user access 306.

Once authorization is confirmed to be authorized, the reiterative data restraint process 310 is initialized. First, the user is prompted to provide a first data entry into a first category textbox 312, such as patient information data on the patient's disease: rheumatoid arthritis. The system then analyzes the data entry to determine whether or not target data is present in the entered data 314. If target data is not present in the entered data, the system prompts the user to provide a next data entry into a next category 318. If target data is present, the system constrains the remaining categories by limiting the possible data entry options 316. The method then determines whether or not the data entry is finished and all the categories are full 317.

If the categories are not full, then the reiterative portion of method begins, as the system then (again) prompts the user to provide a next data entry into a next category 318. The system then (again) analyzes the data entry to determine whether or not target data is present in the entered data 314. If target data is not present in the entered data, the system (again) prompts the user to provide a next data entry into a next category 318. If target data is present, the system (again) constrains the remaining categories by limiting the possible data entry options 316. The method then (again) determines whether or not the data entry is finished and all the categories are full 317.

The reiterative portion of the method repeats until the method determines that all the categories are full, then the data is merged, the categories are compiled, and the letter is finalized 319.

Returning again to the example above, the disease type, “rheumatoid arthritis”, is one example of a target restriction which may be used to further restrain the data entry options into the remaining categories, for example, restraining the total list of professional societies stored to those professional societies which are professionally accredited to provide input on rheumatoid arthritis.

The system then uses that target restriction to restrict wizard tips constructed from accredited professional specialist societies' guidelines such as the guidelines by the American College of Rheumatology. The system then uses the guidelines to target current recommendation treatments to create a target restriction which may then be used to further restrain the patient information 110 to only include treatments which are relevant to rheumatoid arthritis.

Similarly if the guidelines indicated an absence of comorbidity, such as childhood food allergies which have been outgrown as being completely unassociated with rheumatoid arthritis, the present method identifies and flags that irrelevant health care information as flagged (opposed to targeted). All flagged health care information is then labeled as restrained patient information 110 and as such, is prevented from being included in the data that the system accesses.

In this manner, the method is constantly identifying the mandatory components of the selected letter type, categorizing the identified components into a plurality of categories, identifying targets in a first category, restraining the remaining categories based upon the identified target, and reiteratively identifying and restraining all remaining categories upon further identified targets.

### Specific Categories

To facilitate this, the method includes identifying the required components of the mandatory letters. These identified required components are then categorized into different categories depending upon common denominators, such as the associated sources of the data being provided. The term specific within the present specification refers at times to the distinction the system makes between a referenced article that is a target, or otherwise relevant, as opposed to generic articles. Often times, the present inventive method includes a method for distinguishing a feature of relevance of those articles which makes them specific to the overall process opposed to and distinguishable apart from irrelevant articles.

For example, the present invention provides a method to identify a prescribed pharmaceutical as the specific pharmaceutical drug of relevance apart from other pharmaceutical drugs listed. The present invention provides a method to identify a select pharmaceutical manufacturer of relevance as the specific pharmaceutical manufacturer apart from other pharmaceutical manufacturers. The present invention provides a method which identifies the limited purpose as the specific purpose of relevance apart from other purposes.

The present invention provides a method which identifies the degree of compulsory patient confidentiality as the specific amount of patient confidentiality distinguishable from other amounts of patient confidentiality. The present invention provides a method to identify the strictest degree of required HIPAA compliance as the specific degree of compliance of relevance as distinguishable from other degrees of compliance.

The present invention provides a method which identifies the named patient as the specific patient of relevance apart from other patients. The present invention provides a method which identifies the precise history as the specific patient's history of relevance apart from other patients' histories. The present invention provides a method which identifies the unique symptoms as the specific symptoms of relevance apart from other symptoms. The present invention provides tailored tips as the specific tips of relevance apart from other tips. The present invention provides a method which identifies the particular insurance company as the specific insurance company of relevance apart from other insurance companies. The present invention provides a method which identifies the restricted coverage programs as the specific coverage program of relevance apart from other coverage programs.

The present invention provides a secured communications network system as the specific communications network system of the present invention, being distinguishable from other communications network systems, in order to provide a customizable letter template for numerous drugs and devices, yet for one prescribed drug or device per instance.

The method includes restraining results within the national specialist professional societies' guidelines which are provided by professional specialties associations. According to the present method, processing circuitry is configured to filter through all potential national professional societies to identify particular professional societies that are relevant to the patient's condition or disease state. Then, identify one or more alternative databases for identifying a relevant professional guideline. The system queries whether the professional society is indeed accredited to provide professional guidelines (comparing the professional society with a given list of accredited societies to avoid inadvertently obtaining information from AI generated non-accredited sources mimicking professional society guidelines).

If the professional society is accredited, the system 260 is then tasked with obtaining or retrieving the relevant professional guidelines. The system queries whether the guidelines are provided via free public access. If public access is available, then the guidelines are obtained. If not, the system 260 queries the system manager 271 whether to obtain the guideline from the paid subscription information. The system reviews the guidelines and filters the guidelines to find the most current relevant guidelines to patient's specific condition and disease state. The system then populates specifically relevant guideline portions into the wizard tips of the final letter. The wizard further customizes the suggested components of the specifically relevant guideline-based wizard tips by cross-checking with the other identified targets, such as specific insurance requirements.

In some embodiments, the method includes querying electronic health record platforms for a specific patient to obtain specific lab values as well as other key components of patient treatments, history and or chart notes which are all potential requirements of insurance plan requests to prove medical relevance or need. Before the insurance requirements can be used as targets to reduce the other categories, the system must first determine the specific insurance requirements associated with the specific patient's insurance policy. The system identifies the type of insurance coverage, HMO, PPO, HAS, etc. The system analyzes the type of insurance benefits covered by the policy, either medical or pharmaceutical benefits. Once the type of coverage and benefits have been identified and analyzed, the system singles out the associated policy or formulary based upon the pharmaceutical that the physician has selected for the patient. The system obtains access by querying the insurance database 290, fingertip formulary, or paid subscription database that the manufacturer 268 has access to. Once access is granted, the system retrieves the policy.

The present invention overcomes the difficulties of infobesity and infinitely generated combinations by using a method of identifying targets within each of the relevant categories to facilitate a method of reducing the options thereby tailoring a letter for a specific drug, with a specific utilization management criteria, with a verification protocol with an optimization protocol, for a specific pharmaceutical, for a specific purpose, with specific tips to address concerns of the insurers' compliance programs.

Clinical information is necessary to distinguish the disease state and establish the disease attributes.

The present method also includes evaluating the diagnosis and pharmaceutical prescribed against targets identified by the pharmaceutical information 70 obtained from the manufacturer. If the physician attempting to prescribe the pharmaceutical attempts to prescribe a pharmaceutical in a manner that is noncompliant with the manufacturer's indicated use, the method then provides a warning alert and does not allow the request to be completed. In this way, the method ensures that only requests that are compliant with the manufacturer's indicated use are submitted. The method also analyzes and compiles additional criteria to analyze and tailor the final request 200. In this way, the method ensures that the pharmaceutical use is on label according to FDA marketing guidance and requirements.

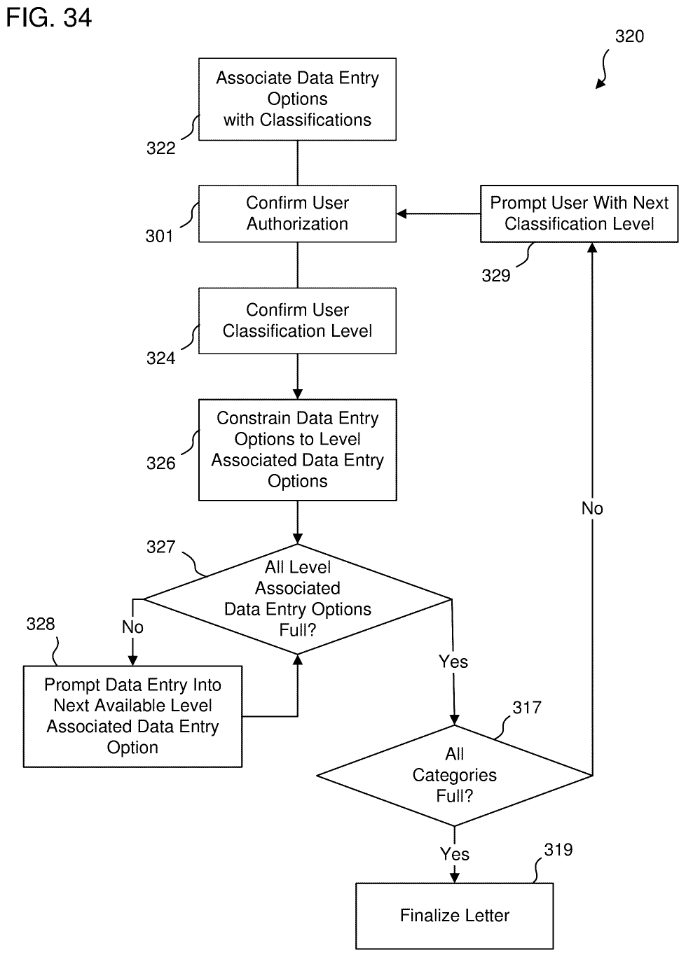

### FIG. 34—Optimization Process & Restraining Classifications

The optimization process further includes further delineating the data entry options by an associated user handling classification 320. Each user handling classification is defined by the necessary qualifications of the individual required for handling the material within that category, e.g., administrative assistances, nurses, pharmaceutical manufacturers, physicians, prescribing physicians, laboratories, patients, security personnel, and other third parties 322.

After the security system verifies the user's authorization to access the present system 301, the user credentials are used to confirm the user's classification level 324. The method then optimizes the data entry options available to the current user by constraining the data entry options to those that are associated with the user's classification level 326.

The method continues to prompt the user for data entry into the next available data entry option associated with the user's classification 328 until all the data entry options associated with the user's classification are full 327. Whereupon the processor determines whether all the categories and all the data entry options are full 317. If further data entry options are empty, yet not associated with the user's classification level, the method prompts the next authorized user with the next highest classification level 329. Once all the data entry options associated with each user's classification levels are full 327, all the categories and all the data entry options are full 317, so the letter can be finalized 319.

In one embodiment, initially during registration and setup 251, the method prompts the office administrator to enter the details regarding the organizations' letterhead 28, users 27, and providers 90 via the secure communication network.

In one embodiment, the manufacturer is funding access to the system. The manufacturer may look at the list of doctors that are likely to need to prescribe the pharmaceutical drug. The manufacturer is then prompted by the system during setup to provide the contact details regarding a plurality of physicians' offices, for each the contact details would include providing the organizations' letterhead 28, authorized users 27, and physicians 90 via the secure communication network. Access credentials would then be provided to the authorized users 27, further reducing the overall workload for physicians and their organizations.

In one embodiment, the manufacturer is funding access to the system. The manufacturer provides a list of pharmaceutical drugs to the system. The system creates a list of physicians that are likely to need to prescribe the pharmaceutical drug. The manufacturer is then queried by the system which physicians to authorize. The system then obtains the contact details for each of the plurality of authorized physicians' offices, for each, the contact details would include providing the organizations' letterhead 28, authorized users 27, and physicians 90 and securely storing those contact details in the secure server memory. Access credentials would then be provided to the authorized users 27, further reducing the overall workload for the manufacturers, physicians, and their respective organizations.

As discussed elsewhere, while the manufacturer may provide information, and while the system may query for information from the manufacturer, the manufacturer may not query or otherwise obtain information from the system. Thus, although the system 260 access in this example is funded by the pharmaceutical company 268, the patient information 110 remains secure, private, and separate from access by the pharmaceutical company 268.

The present method involves assimilating pharmaceutical information 252 which may be done by incorporating pharmaceutical information 130 from databases, again by office administrators. The method may prompt the pharmaceutical company 268 to identify a drug that the manufacturer wants to facilitate physicians to prescribe.

The method may examine publicly accessible sources to provide further pharmaceutical information 70. If publicly accessible sources are available, then the system pursues further pharmaceutical information 70 including targets which are stored securely on the system's server. If not, the method may then cue the manufacturer to provide further pharmaceutical information 70 including targets. The manufacturer may then enter data including further pharmaceutical information 70 including targets which are stored securely on the system's server.

The method includes obtaining all historical patient information 253 including patient demographic information 117, insurance information 120, and patient history 190. This may be done automatically through querying electronic health record platforms. The system may query the electronic health record platforms through application programming interface (API)-set of rules that allow software to communicate. The system may facilitate access to the electronic health record platforms based upon physician's credentials. The system will query whether the system access is necessary. The system will then identify target information specific to the product for indicated use, and restrain system access to that information based upon identified targets. Is information necessary regarding insurance? Is information necessary regarding manufacturer product information? Is information necessary regarding manufacturer guidelines? Only relevant information is thereby allowed to be integrated into the final product 200.

Pharmaceutical companies may have been prompted to provide contact lists. If the pharmaceutical manufacturer was alerted that a patient was denied the drug previously prescribed, access to the system may be granted. The system would start with the patient information, automatically select the doctor that is in that patient's chart.

The present method is reiterative in assessing targets, restraining the applicable categories, then assessing the restrained categories to further provide targets for further restraining other categories. A product has multiple disease states and multiple indications for use. Once the patient information is input, the wizard tips are modified based upon the specific indications that are relevant to that patient. The all the wizard tips are modified based upon the targets identified in the constrained categories of patient information, laboratory results, and the ICD10. Regardless of which category of data is initially input, that data is then analyzed for targets which can then be used to constrain the prompts for further categories. Start putting in patient information, the method alters the constraints for insurance information, and tips. Start putting in insurance information, the method alters the constraints for patient information, and tips for rationales. All of which is being done via instructions on the security enabled server and system that monitors the communication networks involved. The method thereby augments a traditional request, augmenting the clinical expertise that physicians bring to the table, by clarifying and identifying the financial requirements, among other categories of requirements.

Obtaining lab results 254 by gathering those lab results 160 from tests or procedures ordered by the doctor previously, may also be done automatically by querying the laboratories directly and then incorporating that into the patient information by prompting the office admin to grant authorized access to associated laboratory.

### Certifications

As specified elsewhere, the present inventive method and system are built to be HIPAA compliant. The present inventive methods then involve reviewing HIPAA compliance requirements, building the system to be HIPPA compliant, and ensuring continuing HIPPA compliance by audit. Specifically, the inventive method and platform have been HIPAA Audited. The Health Insurance Portability and Accountability Act (HIPAA) is a federal law that protects the privacy of patients' health information and establishes standards for electronic health information. HIPAA was passed by Congress in 1996 and signed into law by President Bill Clinton on Aug. 21, 1996.

The present inventive methods also involve reviewing AICPA SOC compliance requirements, building the system to be AICPA SOC compliant, and ensuring continuing AICPA SOC compliance by audit. Specifically, the inventive method and platform have been AICPA SOC Certified (formerly SAS 70). The American Institute of Certified Public Accountants (AICPA) replaced the SAS 70 report with System and Organization Controls (SOC) reports. The SOC reports include SOC 1, SOC 2, and SOC for Cybersecurity.

The present inventive methods also involve reviewing HITECH compliance requirements, building the system to be HITECH compliant, and ensuring continuing HITECH compliance by audit. Specifically, the inventive method and platform have been HITECH Audited. The Health Information Technology for Economic and Clinical Health (HITECH) Act requires the Office for Civil Rights (OCR) to audit healthcare providers and business associates for compliance with HIPAA. The HITECH Act was established by the American Recovery and Reinvestment Act of 2009.

### LIST OF REFERENCED ELEMENTS

The following reference numbers are adhered to within the specification to refer to those referenced elements within the drawings of the present application.
saved drafts button 1
create new button 2
letterhead/company information button 3
archive button 4
wizard tips/help button 6
pharmaceutical information button 7
user interface 10
initial access page 11
logo A 12
prior user access 13
username 14
password 15
login button 16
new user access 17
sign up button 18
logo B 19
new user registration 20
first name 21
last name 22
email 23
register 24
drafts 26
user details 27
letterhead 28
progress timeline 29
starting page 30
disclaimer 32
instructions 33
template types 34
medical necessity 35
appeals 36
exception 37
prior letters access 38
search bar 39
archive 40
medical necessity patient A 41
appeal patient A 42
exception patient A 43
medical necessity patient B 44
appeal patient B 45
exception patient B 46
medical necessity patient C 47
appeal patient C 48
exception patient C 49
letter wizard settings 50
current logo display 51
change logo 52
choose logo file 53
logo alignment 54
dropdown menu 55
alignment option-left 56
center 57
right 58
wizard tips 60
organizational 61
procedural 62
compatibility 63
validation 64
patient diagnosis criteria 65
clinical guidelines 66
utilization management criteria 67
optimization 68
clinical rationales 69
pharmaceutical information 70
summary 71
warnings 72
contradictions 73
adverse reactions 74
drug interactions 75
indications and usage 76
dosage and administration 77
clinical pharmacology/history 78
clinical studies 79
draft new form 80
date 81
back 82
next 83
back 84
provider information 90
provider first name 91
provider last name 92
provider title 93
provider practice name 94
provider practice address 95
provider practice phone 96
provider signature 97
provider NPI number 98
drafts 100
review draft 101
final draft 102
close 103
save draft 104
create letter 105
patient information 110
first name 111
last name 112
name of policy holder 113
policy holder DOB 114
policy number 115
policy group number 116
electronic health record connection 117
historical information 118
updated information 119
insurance company information 120
contact first name 121
contact last name 122
title 123
company name 124
company address 125
company phone 126
pharmaceutical information 130
prescription name 131
patient diagnosis 140
ICD-10 150
lab results 160
additional details about diagnosis 170
referral information 180
patient history 190
past therapies 191
therapy 192
dosage 193
start date 194
end date 195
description of symptoms 196
rationale 197
rationale for treatment 198
rationales 199
letter 200
greeting 201
statement of purpose 202
summary of diagnosis 203
details 204
rationale for treatment 205
close 206
final product 207
download pdf 208
medical disclaimer clauses 210
clinical consequences of inadequate treatment 211
patient treatment relevance 212
generally accepted standards of care 213
user perspective method 250
registering & setting up 251
obtaining pharmaceutical information 252
obtaining historical patient information 253
obtaining lab results 254
obtaining diagnosis 255
finalizing letter 256
inventive letter creation system 260
HIPAA-compliant server 261
laboratory results (recent) 262
pharmaceutical database 264
electronic health record platforms 265
patient history lab results 266
pharmaceutical manufacturer 268
customized third-party security and service portal 270
dedicated third-party agency interface 271
physician interface 272
nurse interface 273
admin interface 274
physician's network 275
physician's office 278
security 280
security 281
security 282
security 283
insurance agency/paying organization 290
optimization method 300
evaluating user authorization 301
receiving access request 302
assessing whether access is authorized 304
denying access 306
reiterative process by category 310
prompting first data entry 312
identifying the presence of target data 314
constrain data entry into other categories based on target data 316
analyzing whether data entry boxes for all categories are full 317
prompting data entry into next category 318
merging data, compiling category components, finalizing letter 319
reiterative process by classifications 320
associating data entry options with classifications 322
confirming user's classification level 324
constraining data entry options according to classification levels 326
determining whether all associated data entry options are full 327
prompting next available associated data entry option 328
alerting next user with next highest classification level 329

These reference numbers are provided for ease of reference to the accompanying drawings, however, it is anticipated that synonyms for these referenced elements may be used within the specification without detracting from the meaning therein.

### CONCLUSION

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A computer-implemented method for synthesizing a request for pharmaceutical access for a patient comprising:
receiving, by a secured server, a first communication comprising first attempt to access secured data by a first user via a first interface over a secured communications network;
analyzing, by the secured server, first user authorization by comparing first user credentials to authorized user credentials stored on the secured server;
optimizing, by the secured server, a plurality of data entry options available to the first user for entering data by reiteratively:
identifying targets in each of the plurality of categories as key data targets;
prompting a first data entry by the first user into a first data entry option of the plurality of data entry options, the first data entry option being of a first category of a plurality of categories;
evaluating the entered first data entry by comparing the first data entry to the identified targets;
constraining a remainder of the plurality of data entry options associated with a remainder of the plurality of categories according to the first entered identified target;
prompting a next data entry by the first user into a next data entry option of the plurality of data entry options, the next data entry option being of a next category of a plurality of categories;
evaluating the entered next data entry by comparing the next data entry to the identified targets; and
constraining a remainder of the plurality of data entry options associated with a remainder of the plurality of categories according to the next entered identified target.

2. The computer-implemented method according to claim 1, wherein the step of analyzing, by the secured server, the first user credentials, further comprising: determining by the secured server, a classification level of the first user by comparing the first user credentials to a plurality of authorized user classification levels stored on the secured server.

3. The computer-implemented method according to claim 2, wherein the step of optimizing, by the secured server, the plurality of data entry options, further comprising: associating each data entry option of the plurality of data entry options with a classification level of the plurality of authorized user classification levels stored on the secured server.

4. The computer-implemented method according to claim 3, wherein the step of optimizing, by the secured server, the plurality of data entry options, further comprising: populating the first and next data entry options of the plurality of data entry options by comparing first user classification level credentials to the plurality of authorized classification levels associated with each data entry option of the plurality of data entry options.

5. The computer-implemented method according to claim 1, wherein the step of optimizing, by the secured server, the plurality of data entry options, further comprising:
associating each of the plurality of data entry options with at least a first, a second, and a third classification level;
determining a classification level of the first user by comparing first user credentials to a plurality of authorized user classification levels stored on the secured server;
populating the first and next data entry options of the plurality of data entry options by comparing first user classification level credentials to the plurality of authorized classification levels associated with each data entry option of the plurality of data entry options, and constraining the plurality of data entry options to those associated the classification level of the first user; and
prompting a next user with another classification level to begin data entry.

6. The computer-implemented method according to claim 1, wherein the step of optimizing, by the secured server, the plurality of data entry options, further comprising:
automatically populating a first set of the constrained remainder of the plurality of data entry options based upon a second set of the constrained remainder of the plurality of data entry options;
wherein the first set being a first constrained category of the constrained remainder of the plurality of categories; and
wherein the second set being a second constrained category of the constrained remainder of the plurality of categories.

7. The computer-implemented method according to claim 1, wherein the step of optimizing, by the secured server, the plurality of data entry options, further comprising: synthesizing rationale statements from an amalgamation of a first set of the constrained remainder of the plurality of data entry options, a second set of the constrained remainder of the plurality of data entry options, and a third set of the constrained remainder of the plurality of data entry options.

8. The computer-implemented method according to claim 1, wherein each of the plurality of categories is selected from a group of information categories consisting of: pharmaceutical information, patient information, patient diagnosis information, patient history information, provider information, letter purpose information, proprietary statement information, insurance company information, manufacturer information, professional society information, federal drug administration label dosage information, insurance coverage information, security information, compliance information, state regulation information, federal regulation information, and federal guideline information.

9. The computer-implemented method according to claim 2, wherein each of the plurality of classifications is a classification of tiered access levels depending upon the user's identity, and each of the classifications is selected from a group consisting of: nurses, administrative assistants, physician assistants, physicians, prescribing physicians, laboratory assistants, pharmaceutical manufacturer employees, secretaries, and security agents.

10. The computer-implemented method according to claim 2, further comprising:
receiving and transmitting data, by the secured server, via a communications interface connected to a secured communications network from other sources;
recording and storing, by a secured nonvolatile memory, the received and transmitted data;
securing, by a security system, the received, transmitted, recorded, and stored data, the security system having both security hardware and software for securing the secured server and secured communications network; and
continually monitoring, by security interface, the secured server, security system, and secured communications network.

11. A system configured to facilitate selective optimization of a letter for request for a designated prescription drug by an insurance provider, the system comprising:
at least one communication interface configured to transmit data and receive data via a communications network from other sources;
at least one nonvolatile memory, capable of and configured to record and store the received data and transmitted data;
at least one system server capable of and configured to process instructions for the received data and transmitted data; and
at least one security device capable of and configured to secure the at least one communication interface, the at least one nonvolatile memory, the communications network; the at least one nonvolatile memory, and the at least one system server;
whereby the at least one system server capable of and configured to securely process instructions for:
receiving a first communication comprising a first attempt to access secured data by a first user via a first interface over the secured communications network;
analyzing a first user authorization by comparing a first user's credentials to a plurality of authorized user credentials stored on the at least one nonvolatile memory;
optimizing a plurality of data entry options available to the first user for entering data by reiteratively:
identifying targets in each of the plurality of categories as key data targets;
prompting a first data entry by the first user into a first data entry option of the plurality of data entry options, the first data entry option being of a first category of a plurality of categories;
evaluating the entered first data entry by comparing the first data entry to the identified targets;
constraining a remainder of the plurality of data entry options associated with a remainder of the plurality of categories according to the first entered identified target;
prompting a next data entry by the first user into a next data entry option of the plurality of data entry options, the next data entry option being of a next category of a plurality of categories;
evaluating the entered next data entry by comparing the next data entry to the identified targets; and
constraining a remainder of the plurality of data entry options associated with a remainder of the plurality of categories according to the next entered identified target.

12. A computer program product comprising a nonvolatile memory storing computer-readable program code to be executed by at least one processor, the program code comprising instructions configured for:
receiving a first communication comprising a first attempt to access secured data by a first user via a first interface a secured communications network connected to the nonvolatile memory;
analyzing a first user authorization by comparing a first user's credentials to a plurality of authorized user credentials stored on the nonvolatile memory;
optimizing a plurality of data entry options available to the first user for entering data by reiteratively:
identifying targets in each of the plurality of categories as key data targets;
prompting a first data entry by the first user into a first data entry option of the plurality of data entry options, the first data entry option being of a first category of a plurality of categories;
evaluating the entered first data entry by comparing the first data entry to the identified targets;
constraining a remainder of the plurality of data entry options associated with a remainder of the plurality of categories according to the first entered identified target;
prompting a next data entry by the first user into a next data entry option of the plurality of data entry options, the next data entry option being of a next category of a plurality of categories;
evaluating the entered next data entry by comparing the next data entry to the identified targets; and
constraining a remainder of the plurality of data entry options associated with a remainder of the plurality of categories according to the next entered identified target.
